(19) United States
(12) Patent Application Publication
(10) Pub. No.: US 2021/0246019 A1
(43) Pub. Date: Aug. 12, 2021

(54) PEPTIDE/LIPID-ASSOCIATED NUCLEIC ACIDS (PLANA) FOR NUCLEIC ACID DELIVERY

(71) Applicant: Chapman University, Orange, CA (US)

(72) Inventors: Hamidreza Montazeri Aliabadi, Rancho Santa Margarita, CA (US); Keykavous Parang, Irvine, CA (US); Ryley Hall, Tacoma, WA (US); Abdulaziz Alasmari, Orange, CA (US)

(21) Appl. No.: 17/170,785

(22) Filed: Feb. 8, 2021

Related U.S. Application Data
(60) Provisional application No. 62/971,593, filed on Feb. 7, 2020.
(60) Provisional application No. 63/071,588, filed on Aug. 28, 2020.

(51) Int. Cl. B82Y 5/00; A61K 47/54; A61K 47/69; A61K 47/26; A61K 47/18

(52) U.S. Cl. CPC B82Y 5/00; A61K 47/543; A61K 47/18; A61K 47/26; A61K 47/6929

(57) ABSTRACT

Disclosed are compositions and methods for delivering nucleic acids and other substances into living cells. The compositions constitute a delivery system comprising a peptide component with hydrophobic and positively-charged portions, and a lipid component. Any nucleic acid can be delivered including siRNA, mRNA, and DNA. The delivered nucleic acid can have therapeutic or prophylactic effect.

### RELATED APPLICATIONS

This application claims the benefit of priority to U.S. Provisional Application Nos. 62/971,593 filed Feb. 7, 2020, and 63/071,588 filed Aug. 28, 2020; the entire contents of which are each incorporated by reference herein.

### FIELD

The field of the invention is delivery of nucleic acids (DNA, plasmids, oligos, small interfering RNAs [siRNA], small hairpin RNA [shRNA], microRNAs [miRNA], Clustered Regularly Interspaced Short Palindromic Repeats [CRISPR]) to living cells in vitro and in vivo.

### BACKGROUND

The background description includes information that may be useful in understanding the present invention. It is not an admission that any of the information provided herein is prior art or relevant to the presently claimed invention, or that any publication specifically or implicitly referenced is prior art.

It can be useful to deliver nucleic acids into cells for a variety of purposes. Commonly, nucleic acid delivery is an approach to suppress (or enhance) the expression of a protein temporarily or permanently. The suppression of protein expression (also known as silencing) is performed by delivering double-stranded siRNAs, shRNA, miRNA, or CRISPR/Cas9. The siRNAs contain a passenger and a guide strand. The guide strand is incorporated into the RNA-induced silencing complex (RISC), while the passenger strand is degraded. The guide strand acts as a complementary sequence to the messenger RNA, and therefore, binds to the targeted mRNA, which triggers the Argonaute 2 (an essential catalytic protein in the RISC) to cleave the mRNA into small pieces, which will be degraded rapidly by RNases. This process is known as the post-transcriptional gene silencing and results in temporary silencing of the expression of a targeted protein. This complex intervenes in the degradation process of the siRNA sense strand and utilizes the preserved anti-sense strand to identify the complementary sequences of mRNA. shRNA and miRNA use the same RISC system; however, they are delivered as plasmids that are incorporated into the chromosome and result in permanent shRNA or miRNA production, and therefore, permanent silencing of the targeted protein. CRISPR-Cas9 is a new RNA-guided gene editing tool that is comprised of a nuclease (Cas9) and a single-stranded guide RNA (sgRNA), which can permanently remove the targeted gene from DNA, change the DNA sequence, or promote insertion of an exogenous DNA sequence. There are many variations of CRISPR known which use different nucleases and can be adapted for still other purposes. The enhancement of protein expression is usually performed by delivering circular pieces of DNA, known as plasmids that are incorporated into the chromosome and are transcribed as other protein-expressing pieces of the genome.

Clinical evaluation of nucleic acids has been challenging due to their limited cellular uptake (because of large size and negatively charged phosphate groups in their structure), off-target effects, and rapid enzymatic degradation in vivo. Even though double stranded nucleotides are generally more stable, RNA is still extremely susceptible to enzymatic degradation in biological settings. Also, nucleic acids are readily eliminated from the body through glomerular filtration. The nuclease degradation of nucleic acids can occur rapidly anywhere in the body, which inactivates the molecule. Finally, the negatively charged and hydrophilic nature of nucleic acid structures have minimal interaction with the cell membrane (which is also negatively charged), and therefore, the cellular internalization is negligible.

Thus, compositions and methods of improved delivery of nucleic acids to cells are needed.

### SUMMARY

Disclosed herein is a multi-component nucleic acid delivery system called peptide/lipid-associated nucleic acids (PLANA) and associated methods of making and using PLANA. As the name implies, PLANA has three basic components: a peptide component, a lipid component, and the nucleic acid to be delivered. This mixture of components can be formed into nanoparticles to facilitate delivery and uptake of the so formulated nucleic acid. Some embodiments are a pharmaceutical composition comprising the PLANA.

One aspect is a formulated nucleic acid comprising a mixture of 1) aqueous nucleic acid, 2) lipid dissolved in an alcohol or other organic solvent, and 3) peptide with a positively charged region and a hydrophobic portion; a PLANA. In some embodiments, the formulation is formed into nanoparticles; PLANA nanoparticles.

One aspect is a method of making the formulated nucleic acid comprising providing an aqueous solution of the nucleic acid component, providing an organic (for example, alcoholic) solution of a lipid component, and mixing them together. In some embodiments, the nucleic acid in the aqueous solution is at a concentration in the range of 10 to 25 mg/ml, or 15 to 20 mg/ml, for example, about 17.5 mg/ml. In some embodiments, the aqueous and alcoholic portions are mixed in a ratio of 3:1 v/v. In some embodiments, the peptide is added to the aqueous portion. In some embodiments, the peptide is added to the organic portion, that is, the lipid solution. In some embodiments, the organic solvent is an alcohol. In some embodiments, the alcohol is ethanol. In some embodiments, the PLANA formulation is formed into nanoparticles. In some embodiments, the nanoparticles are obtained by extrusion. In some embodiments, the PLANA nanoparticles have a diameter around 100 nm. In some embodiments, the PLANA nanoparticles are dialyzed (whereby the organic solvent is removed).

One aspect is a method of delivering a nucleic acid into cells by contacting a PLANA formulation or PLANA nanoparticles with the cells. In some embodiments, the contacting takes place in vitro. In some embodiments, the contacting takes place in vivo. In some embodiments, the PLANA formulation or PLANA formulation nanoparticle comprises one of more nucleic acids. In some embodiments, the cell is contacted with one or more PLANA formulations (in the form of nanoparticles or otherwise), wherein each PLANA formulation comprises a different nucleic acid that the other(s).

With respect to any of the foregoing aspects, in some embodiments, the nucleic acid is a small interfering RNA (siRNA). In some embodiments, the nucleic acid is a hairpin RNA, such as a short hairpin RNA (shRNA). In some embodiments, the nucleic acid is a microRNA (miRNA). In some embodiments, the nucleic acid is a messenger RNA (mRNA). In some embodiments, the nucleic acid is an antisense RNA. In some embodiments, the nucleic acid is a single-stranded guide RNA (sgRNA) for a CRISPR system. In some embodiments, the nucleic acid is an mRNA or DNA encoding one or more components of a CRISPR system. In some embodiments, the nucleic acid is a DNA molecule. In some embodiments, the DNA molecule encodes and can express any of the mentioned RNA molecules. In some embodiments, the DNA molecule is a plasmid. In some embodiments, the nucleic acid is an oligonucleotide. In some embodiments, the nucleic acid is transiently present in the cell. In some embodiments, the nucleic acid persists episomally. In some embodiments, the nucleic acid becomes integrated into the cell's genome.

One aspect is a method of gene silencing comprising contacting a cell with a PLANA formulation or PLANA nanoparticles, wherein the nucleic acid is a siRNA, a shRNA, a miRNA, a sgRNA, or an mRNA or DNA encoding one or more components of a CRISPR system.

With respect to any of the foregoing aspects, the peptide component comprises a positively charged portion to bind to the (negatively charged) nucleic acid, and a hydrophobic portion to facilitate interaction with the cell membrane and improve cellular uptake. In some embodiments, the peptide component is a linear peptide. In some embodiments, the peptide component is a cyclic peptide. In some embodiments, the peptide component is a cyclic/linear hybrid peptide (as used herein, a hybrid peptide); that is, a cyclic peptide with a linear peptide covalently attached to it. In some embodiments, the hydrophobic portion of the peptide comprises a ring of hydrophobic amino acids. In some embodiments of a hybrid peptide, the cyclic portion is hydrophobic and the linear portion is positively charged. In alternate embodiments of a hybrid peptide, the cyclic portion is positively charged and the linear portion is hydrophobic. In some embodiments, the hydrophobic portion comprises a fatty acyl conjugation. In some embodiments, the hydrophobic portion comprises a chain of hydrophobic amino acids. In some embodiments, the hydrophobic portion comprises a chain of tryptophan residues. In some embodiments, the positively charged portion of the peptide comprises arginine and/or lysine residues. In some embodiments, the positively charged portion of the peptide comprises a ring of arginine and/or lysine residues.

With respect to any of the foregoing aspects, the lipid component can comprise a mixture of lipids. The lipids may be neutral or positively charged. Some embodiments also comprise negatively charged lipids. Some embodiments comprise 1,2-dioleoyl-3-trimethylammonium-propane (DOTAP; optionally a chloride salt), cholesterol, phosphatidylcholine, 1,2-dioleoyl-sn-glycero-3-phosphoethanolamine (DOPE, a neutral lipid) or any combination thereof. Some embodiments specifically include one or more of these lipids. Some embodiments specifically exclude one or more of these lipids. Some embodiments include only DOTAP, cholesterol, phosphatidylcholine, and DOPE in the lipid component. Some embodiments include only cholesterol, phosphatidylcholine, and DOPE in the lipid component.

One aspect is a method of inhibiting a coronavirus infection comprising contacting an infected cell with PLANA nanoparticles comprising siRNA targeting coronavirus envelope (E) and/or spike protein (S) genes.

### BRIEF DESCRIPTION OF THE DRAWINGS

FIGS. 1A-B depict the structure of fatty acyl-conjugated peptides LP-C16 and LP-C18 in linear form. FIG. 1A shows the peptides with free N-terminal amino groups. FIG. 1B shows LP-C18 with an N-acetyl cap, as used in the Examples described herein.

FIG. 2 depicts the structure of fatty acyl-conjugated peptides CP-C16 and CP-C18 in cyclic form.

FIG. 3 depicts the structure of cyclic/linear hybrid peptides comprising a ring of arginine residues and one lysine residue conjugated through its ε-amino group to a linear chain of tryptophan residues; specifically, [R₅K]W₅, [R₆K]W₆, and [R₅K]W₇.

FIG. 4 depicts the structure of further fatty acyl-conjugated linear and cyclic peptides.

FIG. 5A presents a synthetic scheme for cyclic peptide CP-C18.

FIG. 5B presents a synthetic scheme for linear peptide LP-C18.

FIG. 5C presents a synthetic scheme for cyclic/linear hybrid peptide [R₅K]W₇.

FIGS. 6A-B depict dynamic light scattering of PLANAs: A Malvern Nano ZS Zetasizer was used to measure the size of the PLANA nanoparticles prepared with fatty acyl-conjugated and hybrid peptides (FIG. 6A), as well as the surface electrical charge ( [symbol] -potential) of the two categories of peptides (FIG. 6B). The bar graphs represent the average (n=3) values and error bars show the standard deviation for each study group.

FIGS. 7A-C depict scanning electron microscope (SEM) images of selected PLANA formulations: LP-C18 24 h. incubation (FIG. 7A); PLANAs (LP-C18/siRNA) No incubation time (FIG. 7B); and CP-C18 24 h. incubation (FIG. 7C).

FIG. 8 depicts transmission electron microscope (TEM) images of selected PLANA formulations: CP-C18 24 hr incubation (left panel) and PLANAs (CP-C18+siRNA) no incubation (right panel).

FIGS. 9A-C present images of SDS-PAGE gels comparing electrophoretic mobility of PLANA formulations compared to free peptide loaded in amounts equivalent to 10%, 20%, and 100% of the peptide incorporated in the PLANA formulation. FIG. 9A depicts a PLANA formulation prepared with peptide included in the aqueous phase FIG. 9B depicts a PLANA formulation prepared with peptide included in the alcoholic phase. FIG. 9C depicts a [R₅K]W₅ formulation prepared with cyclic/linear hybrid peptide included in the aqueous phase.

FIG. 10 depicts siRNA encapsulation efficiency as studied via quantification of free siRNA using a SYBR Green Dye II exclusion assay. Bar graphs indicate the mean value (n=3), and the error bars represent standard deviation.

FIGS. 11A-B depict siRNA release profiles. The amount of siRNA released from the dialysis bag into the receiving phase was quantified at pre-determined time points (0.5, 2, 4, 24, 48, and 72 hours) for fatty-acyl conjugated (FIG. 11A) and hybrid peptides (FIG. 11B) as compared to free siRNA. Each data point represents the mean values (n=3) and the error bars indicate the standard deviation.

FIG. 12 depicts release profiles of the hybrid peptides incorporated into PLANA D formulations. The percentage of peptide released from the dialysis bag into the receiving phase was quantified at pre-determined time points (0.5, 2, 4, 24, 48, and 72 hours). Each data point represents the mean values (n=3), and the error bars indicate the standard deviation.

FIGS. 13A-B depict the size (FIG. 13A) and surface electrical charge (FIG. 13B) of selected fatty acyl-containing PLANA formulations before and after the release study. Bar graphs and error bars represent the mean values (n=3) and standard deviations, respectively.

FIGS. 14A-F present cell viability measurements after exposure to formulation components as compared to no treatment (normal saline) for PLANA formulations prepared using LP-C18 (FIG. 14A), CP-C18 (FIG. 14B), [R₅K]W₅ (FIG. 14C), [R₆K]W₆ (FIG. 14D), and [R₅K]W₇ (FIG. 14E) in triple negative cell line MDA-MB-231. The concentrations represent equivalent siRNA final concentration in cell culture media. The toxicity of the designed delivery system was also evaluated for PLANA D formulations in non-cancerous human lung, myocardium, hepatocytes, and epidermal keratinocytes, in concentrations representing equivalent siRNA final concentration of 50 and 100 nM (FIG. 14F). Bars and error bars represent mean and standard deviation (n=3) values.

FIGS. 15A-E present data on cellular internalization of siRNA using various formulations assessed by flow cytometry. The percentage of the cells positive for siRNA fluorescence signal was quantified for LP-C18 (FIG. 15A), CP-C18 (FIG. 15B), [R₅K]W₅ (FIG. 15C), [R₆K]W₆ (FIG. 15D), and [R₅K]W₇ (FIG. 15E) containing PLANA formulations in MDA-MB-231 cells. The bars and error bars in each panel represent the percentage of cells positive for siRNA signal and represent the mean and standard deviation values (n=3).

FIG. 16 depicts the mean fluorescence values from flow cytometry of MDA-MB-231 cells exposed to PLANA D formulations incorporating different peptides, as indicated.

FIGS. 17A-B present color separations, and merged images, of multicolor fluorescence stained cells treated with AF488-labeled siRNA alone or 5 PLANA D formulations. The left-most column show staining of nuclei with DAPI. The center-left column shows fluorescence from the AF488-labeled siRNA. The center-right column shows staining of the cell membrane with Texas Red Phalloidin. The right-most column shows merger of the colors. FIG. 17A shows the result for AF488-labeled siRNA alone, Lipofectamine® alone, and PLANA D formulations of LP-C18 and CP-C18. FIG. 17B shows the results for PLANA D formulations of [R₅K]W₅, [R₆K]W₆, and [R₅K]W₇.

FIGS. 18A-B present data showing protein silencing by siRNA delivered by different reagents. The expression of RPS6KA5 (FIG. 18A) and Src (FIG. 18B) was quantified in MDA-MB-231 cells after siRNA delivery via selected PLANA E formulations, compared to No Treatment (NT) and Lipofectamine® as negative and positive controls, respectively. GAPDH was used as an endogenous protein to normalize the band intensities.

FIGS. 19A-D depict the viability of uninfected (NV) and hCoV-229E-infected (virus) cells subjected to various treatments. The treatments were: siRNA delivered with 12-18-6A polymer (FIG. 19A), siRNA delivered with PLANA (FIG. 19B), siRNA delivered with 12-18-9A polymer (FIG. 19C), and remdesivir (FIG. 19D).

FIG. 20 presents data showing that targeting S and E proteins in coronavirus 229E via PLANAs completely eliminated plaques seen in no treatment group (arrows; upper panel) as compared to the treatment group (lower panel).

### DETAILED DESCRIPTION

Disclosed herein is a multi-component nucleic acid delivery system called peptide/lipid-associated nucleic acids (PLANA) and associated methods of making and using PLANA. As the name implies, PLANA has three basic components: a peptide component, a lipid component, and the nucleic acid to be delivered. This mixture of components can be formed into nanoparticles to facilitate delivery and uptake of the formulated nucleic acid. Some embodiments are a pharmaceutical composition comprising the PLANA.

Using siRNA as a model, the inventors have developed a multi-component carrier based on the incorporation of siRNA and the designed fatty acyl-conjugated and cyclic/linear hybrid peptides into a lipid nanoparticle to form PLANAs for siRNA delivery into living cells. A multi-component hydrophobic nanoparticle can efficiently incorporate the amphiphilic peptides, encapsulate nucleic acid (due to interionic interaction with the peptide), protect the nucleic acid from early enzymatic interaction, efficiently internalize the cargo into the cells, and release it in a timely manner for efficient delivery. Delivery of short interfering RNAs (siRNAs) remains a major challenge in the development of RNA interference therapeutics. siRNA is a class of double-stranded RNA molecules that are 20-25 base pairs long. An optimal siRNA delivery system for in vivo use must facilitate cellular uptake and enhance its activity. Second, it must direct the siRNA toward the target tissue effectively. However, the challenges of delivering siRNA are not unique, but rather apply to the delivery of nucleic acids generally.

Formulations of PLANAs represent a new class of nucleic acid delivery systems. The structures of these series of formulations are different from those of current delivery systems. The incorporation of specifically designed amphiphilic peptides into a multi-component delivery system has not been previously pursued or described. Previous nucleic acid delivery systems have not made use of peptides that are hydrophobic, yet carry positive charge. Neither have the disclosed combinations of peptide and lipid been utilized. The methodology for preparation of PLANAs, their physical characteristics, in vitro siRNA and peptide incorporation, siRNA release profile, cytotoxicity, siRNA delivery, and silencing potential are described herein.

The disclosed compositions can include three classes of peptides, linear, cyclic, and/or hybrid linear/cyclic peptides containing natural or non-natural positively charged amino acids, and hydrophobic residues or added side-chains for use as nucleic acid delivery systems, and incorporation of these peptides in a multi-component nanoparticles to enhance the efficiency of the lipid nanoparticles. This nucleic acid delivery system is distinct since it includes an innovative nanoparticle that encompasses all the benefits of cell-penetrating peptides, hydrophobic nature of neutral lipids, lipid bilayer forming lipids, and the potential for PEGylation and adding targeting moieties for active in vivo targeting.

One should appreciate that the disclosed techniques provide many advantageous technical effects including efficient and direct introduction of nucleic acids (which could include genetic material, oligonucleotides, and CRISPR components) into living cells.

The nucleic acid component of PLANAs interacts with the other components primarily through their negatively charged phosphate backbones. As this feature is universal, PLANAs can be used to deliver any type of nucleic acid used for any purpose. In some embodiments, the nucleic acid is a small interfering RNA (siRNA). In some embodiments, the nucleic acid is a hairpin RNA, such as a short hairpin RNA (shRNA). In some embodiments, the nucleic acid is a microRNA (miRNA). In some embodiments, the nucleic acid is a messenger RNA (mRNA). In some embodiments, the nucleic acid is an antisense RNA. In some embodiments, the nucleic acid is a single-stranded guide RNA (sgRNA) for a CRISPR system. In some embodiments, the nucleic acid is an mRNA or DNA encoding one or more components of a CRISPR system. In some embodiments, the nucleic acid is a DNA molecule. In some embodiments, the DNA molecule encodes, and can express, any of the mentioned RNA molecules. In some embodiments, the DNA molecule is a plasmid. In some embodiments, the nucleic acid is transiently present in the cell. In some embodiments, the nucleic acid persists episomally. In some embodiments, the nucleic acid becomes integrated into the cellular genome. In general, delivered RNA will persist in the cell for only a limited period of time, and thus its direct effect will be transient. For longer term effects, a DNA molecule encoding the RNA in an expressible form can be delivered. A non-episomal, non-integrating DNA will generally provide longer-term presence of the RNA than direct delivery of the RNA, but will not be permanent, especially in dividing cells. An episomal or integrating DNA can provide a permanent effect.

The peptide component of PLANAs provides the primary positive charge that mediates interaction with the nucleic acid. The peptide component also provides a hydrophobic portion that mediates interaction with the lipid component and with the cell membrane that can both directly and indirectly (through the lipid component) facilitate cellular uptake.

The positively charged portion of the peptide component comprises positively charged amino acids, such as arginine, lysine, histidine diaminopropionic acid (Dap), and diaminobutyric acid (Dab). In some embodiments, the positively charged portion of the peptide consists of arginine and lysine residues. In some embodiments, positive charge is provided by the arginine residues and the ε-amino group of the lysine residues serves as a point of attachment for the hydrophobic portion of the peptide. In some embodiments, the positively charged portion of the peptide comprises a ring of arginine and/or lysine residues. In other embodiments, the peptide is linear.

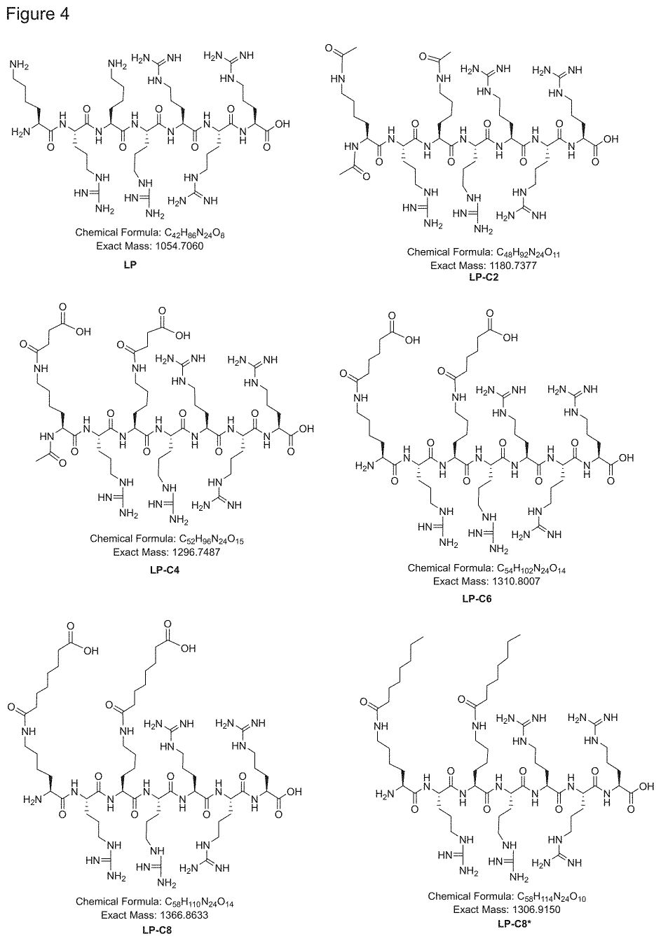

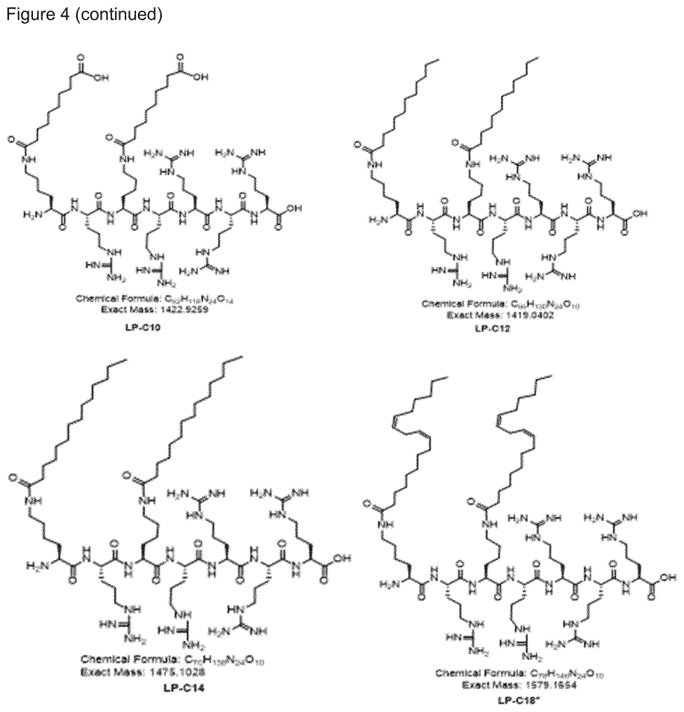

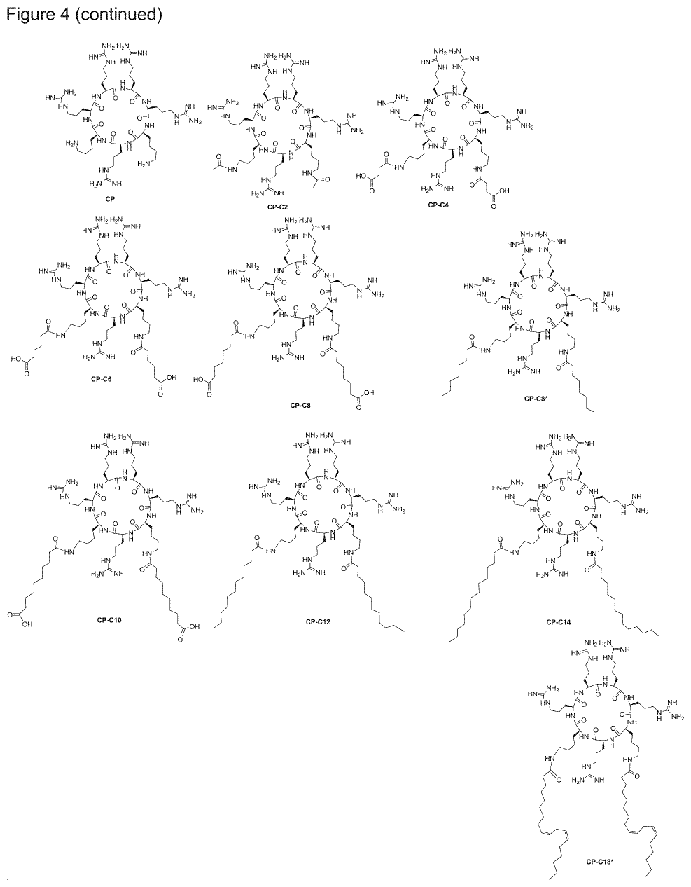

The hydrophobic portion of the peptide can be either a chain of hydrophobic amino acids, for example, tryptophan, or a fatty acyl group, as, for example, from a C18 fatty acid. In some embodiments, the peptide comprises one hydrophobic group, for example, a chain of tryptophan residues. In some embodiments, the peptide comprises multiple hydrophobic groups. In some embodiments, the peptide comprises two hydrophobic groups, for example two fatty acyl chains. In some embodiments, the hydrophobic group(s) is conjugated to the charged portion of the peptide by attachment to the ε-amino group of a lysine residue. In some embodiments, the fatty acyl chain is saturated. In other embodiments, the fatty acyl chain contains one or more unsaturated bonds (see, for example, LP-C18* and CP-C18* in FIG. 4). In some embodiments, fatty acyl chain length is in the range of C12 to C24, C14 to C22, or C16 to C20. In some embodiments, fatty acyl chain length is C18.

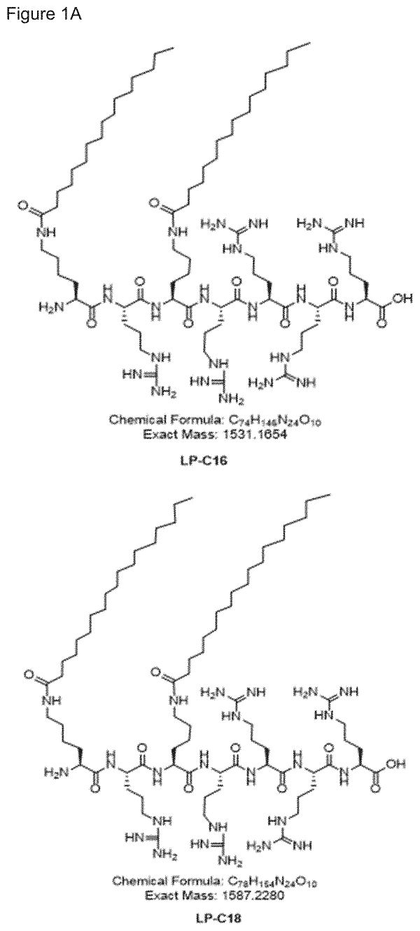

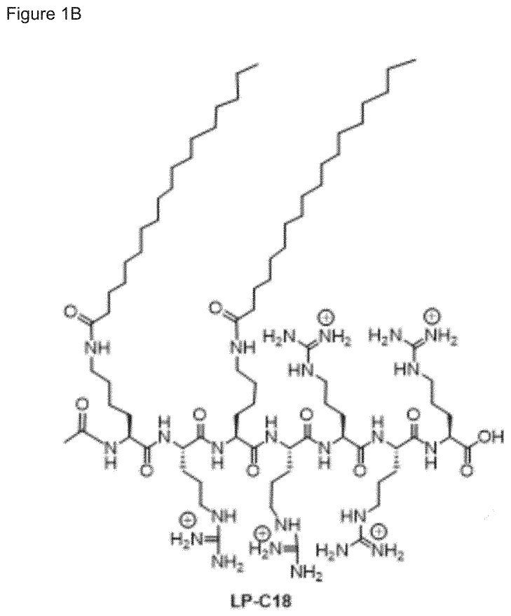

As can be appreciated from FIGS. 1-4, the attachment of the hydrophobic portion to a lysine side-chain gives the peptide a branched structure. In some embodiments, multiple branches extend in the same general direction.

The peptide component of the PLANA can be designed to incorporate additional features. In some embodiments, the peptide comprises a non-peptide bond linking adjacent amino acids. In some embodiments, the non-peptide bond is selected from the group consisting of a thioamide bond, an N-methyl bond, and a CH2-NH bond. In some embodiments, the peptide comprises of a plurality of a positively charged amino acids X and a plurality of a hydrophobic amino acids Y, wherein positively charged amino acids X and hydrophobic amino acids Y are coupled to one another in an alternating or block fashion. In some embodiments, a plurality of a positively charged amino acids X coupled to one another in a first segment and a plurality of a hydrophobic amino acids Y coupled to one another in a second segment, wherein the first segment and the second segment are coupled to one another. In some embodiments, the peptide comprises a modification to a side chain of a charged amino acid X or a hydrophobic amino acid Y.

In some embodiments, the peptide comprises a plurality of cysteine residues (C). In some of those embodiments, the peptide is a cyclic peptide comprising a disulfide bond between two of the plurality of cysteine residues, wherein the disulfide bond forms part of the cyclic structure. In some embodiments, (with X still representing a charged amino acid and Y representing a hydrophobic amino acid, and K represents lysine) the peptide is linear and has a structure (Y)ₙCXₙC(Y)n or (X)ₙCYₙC(X)ₙ. In some embodiments, the peptide is linear and has a structure (X)ₙ(KYₙK)(X)ₙ. In some embodiments, the peptide is linear and has a structure (X)ₙ(Yₙ)(X). In some embodiments, the peptide is a hybrid cyclic/linear peptide and has a structure (Y)ₙ[CXₙC](Y)ₙ, a hybrid cyclic-linear peptide (X)ₙ[CYₙC](X)ₙ. In some embodiments, the peptide is a hybrid cyclic/linear peptide and has a structure (Y)ₙ[KXₙK](Y)ₙ. In some embodiments, the peptide is a hybrid cyclic/linear peptide and has a structure (X)ₙ[KYₙK](X)ₙ. In some embodiments, the peptide is linear and comprises a plurality of hydrophobic residues coupled to two chains of hydrophilic residues through two cysteine residues. In some embodiments, the peptide is linear and comprises a plurality of hydrophilic residues coupled to two chains of hydrophobic residues through two cysteine residues. In some embodiments, the peptide is cyclic and comprises a plurality of hydrophobic residues coupled to two chains of hydrophilic residues through two cysteine residues. In some embodiments, the peptide is cyclic and comprises a plurality of hydrophilic residues coupled to two chains of hydrophobic residues through two cysteine residues. In some embodiments, the peptide is linear and comprises a plurality of hydrophobic residues coupled to two chains of hydrophilic residues through two lysine residues. In some embodiments, the peptide is linear and comprises a plurality of hydrophilic residues coupled to two chains of hydrophobic residues through two lysine residues. In some embodiments, the peptide is cyclic and comprises a plurality of hydrophobic residues coupled to two chains of hydrophilic residues through two lysine residues. In some embodiments, the peptide is cyclic and comprises a plurality of hydrophilic residues coupled to two chains of hydrophobic residues through two lysine residues.

In some embodiments, when the hydrophobic portion comprises a chain of hydrophobic amino acids, wherein the chain length is 4, 5, 6, 7, or 8 residues. In some embodiments, the residues are all the same amino acid. In alternative embodiments, the hydrophobic chain comprises a mixture of hydrophobic amino acids. In some embodiments, the hydrophobic amino acid is selected from tryptophan, phenylalanine, isoleucine, leucine, norleucine, valine, norvaline, p-phenyl-L-phenylalanine (Bip), 3,3-diphenyl-L-alanine (Dip), 3(2-naphthyl)-L-alanine (Nal), 6-amino-2-naphthoic acid, 3-amino-2-naphthoic acid, 1,2,3,4-tetrahydronorharmane-3-carboxylic acid, 1,2,3,4-tetrahydro-3-isoquinolinecarboxylic acid (Tic-OH), 1,2,3,4-tetrahydro-3-isoquinolinecarboxylic acid, a modified D- or L-tryptophan residue, an N-alkyl tryptophan, an N-aryl tryptophan, a substituted d- or 1-tryptophan residue, 5-hydroxy-L-tryptophan, 5-methoxy-L-tryptophan, 6-chloro-L-tryptophan, a fatty amino acid having a formula NH₂-(CH₂)x-COOH, wherein x=1-20, or an N-heteroaromatic amino acid. In some embodiments, the hydrophobic amino acid is tryptophan.

In some embodiments, when the hydrophobic portion comprises a chain of hydrophobic amino acids, the positively charged portion of the peptide is a cyclic peptide, while the hydrophobic chain is linear. These peptides are referred to as cyclic/linear hybrid peptides, or hybrid peptides.

In some embodiments, the peptide amino acids are L-amino acids. In some embodiments, they are R-amino acids. In some embodiments, a position-by-position mixture of L- and R-amino acids is used. In some embodiments, a mixture of L- and R-amino can be present at one or more, or all, positions.

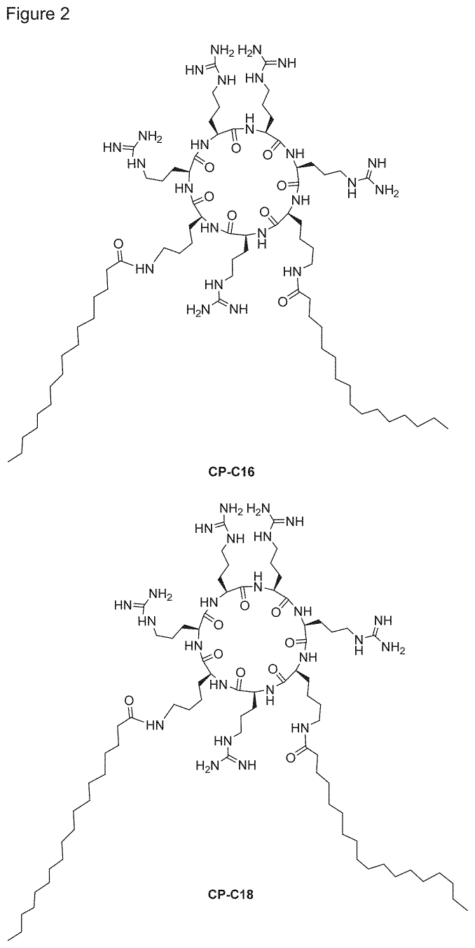

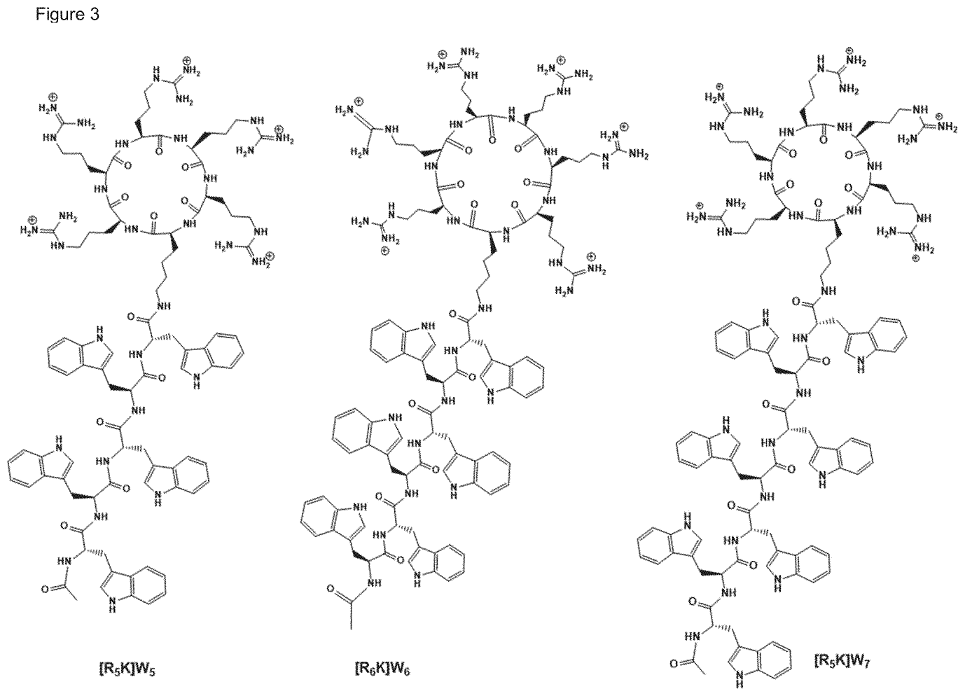

Exemplary peptides to be incorporated into PLANA formulations representing three different categories of peptides include: LP-C16 and LP-C18 as linear fatty acyl-conjugated peptides (FIG. 1), CP-C16 and CP-C18 as cyclic fatty acyl-conjugated peptides (FIG. 2), and [R₅K]W₅, [R₆K]W₆, and [R₅K]W₇ as cyclic/linear hybrid peptides (FIG. 3). Additional examples of suitable peptides are shown in FIG. 4.

In some embodiments, the cyclic peptide ring is closed with a peptide bond, for example, by forming a peptide bond between the N-terminal α-amino group and C-terminal carboxyl group of a linear precursor. In other embodiments, the ring comprises a non-peptide bond. In some embodiments, the non-peptide bond is a cysteine bond.

The lipid component can be selected from a wide variety of neutral (e.g., DOPE), cationic (e.g., DOTAP), and anionic lipids and different compositions, including, for example, cholesterol and phosphatidyl choline. In various embodiments, the lipid component may include or exclude one or more of these lipids, and may or may not comprise other lipids. Due to the positive charge of the peptide component, cationic lipids are not required. The absence of cationic lipid leads to PLANA with lower surface charge. This is advantageous since many studies have demonstrated a higher toxicity for cationic lipids and a higher risk of failure in targeting due to interactions of these lipids with negatively charged molecules in blood circulation. In some embodiments, the PLANA reagent (that is, not including the nucleic acid) contains a molar fraction of peptide in the range of 0.006 to 0.05, for example 0.00625 or 0.0425. The lower molar fraction being useful for PLANAs incorporating cationic lipid and the higher molar fraction being needed in PLANAs lacking cationic lipid. In some embodiments, the molar fraction of peptide plus cationic lipid is equal to the molar fraction of each of the other lipids in the PLANA. In some embodiments lacking cationic lipid, the molar fraction of each lipid is equal. In some embodiments, the molar fraction of one or more lipids is above the mean, and one or more is below the mean.

In some embodiments, the lipid component is made up of DOTAP, cholesterol, phosphatidylcholine, and DOPE. In some embodiments the molar fraction of cholesterol, phosphatidylcholine, and DOPE is 0.25 and the molar fraction of DOTAP plus peptide is 0.25 (for example, 0.244 and 0.00625, respectively; PLANA A).

In some embodiments, the lipid component is made up of cholesterol, phosphatidylcholine, and DOPE. In some embodiments, the molar fraction for each of cholesterol, phosphatidylcholine, and DOPE is equal, for example, 0.319 (with 0.0425 peptide; PLANA B). In some embodiments, the amount of cholesterol is reduced and the amount of phosphatidylcholine is increased relative to the mean, for example, 0.128 cholesterol and 0.510 phosphatidylcholine (with 0.319 DOPE and 0.0425 peptide; PLANA C). In some embodiments, the amount of cholesterol and phosphatidylcholine are reduced and the amount of DOPE is increased relative to the mean, for example, 0.128 cholesterol, 0.256 phosphatidylcholine and 0.574 DOPE (with 0.0425 peptide; PLANA D).

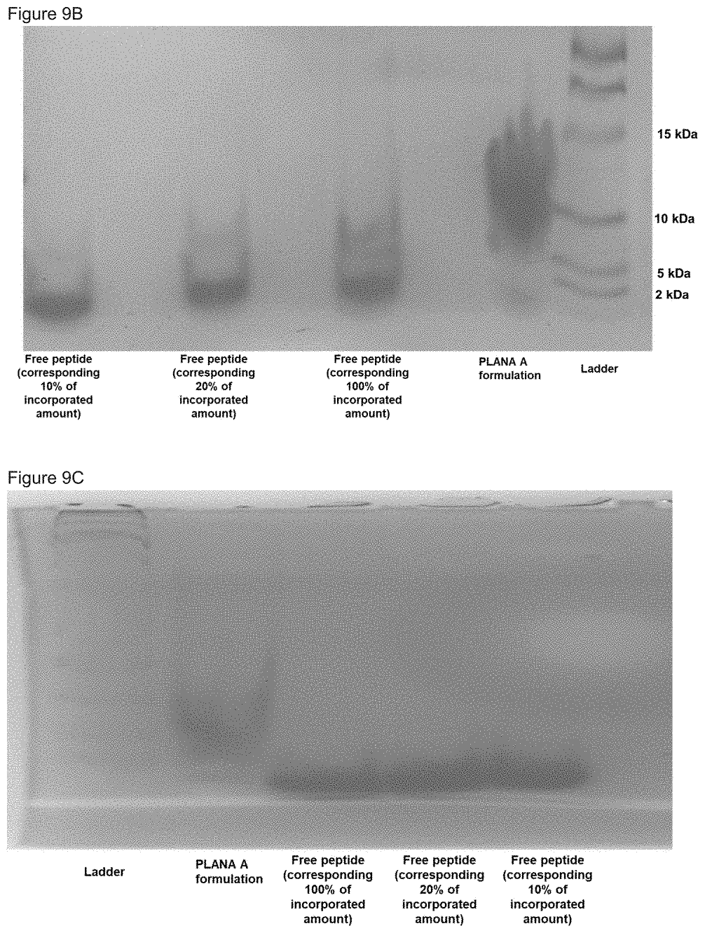

To make the formulated nucleic acid, a PLANA, an aqueous solution of the nucleic acid component and an organic (for example, alcoholic) solution of a lipid component are mixing together. In some embodiments, the organic solvent is an alcohol. In some embodiments, the alcohol is ethanol. In some embodiments, the aqueous and alcoholic portions are mixed in a ratio of 3:1 v/v. In some embodiments, the peptide component, which is amphipathic, is added to the aqueous solution. In other embodiments, the peptide component is added to the organic component. Better incorporation of the peptide component has been observed when adding the peptide to the aqueous portion (see Example 5 and FIGS. 9A-C). In some embodiments, the nucleic acid in the aqueous solution is at a concentration in the range of 10 to 25 mg/ml, or 15 to 20 mg/ml, for example, about 17.5 mg/ml. In some embodiments, the nucleic acid is a siRNA. In some embodiments, the siRNA in the aqueous solution is at a concentration of 1000 nM.

In some embodiments, the PLANA is formed into nanoparticles. Nanoparticles may be formed by extrusion of the PLANA formulation through a filter. In some embodiments the filter is a 100 nm filter. In some embodiments, the PLANA formulation is passed through the filter a plurality of times. In one embodiment, the PLANA formulation is passed through the filter 50 times. In some embodiments, the nanoparticles are in a range of about 50-1000 nm, about 80 to 200 nm, or about 100-150 in diameter. In some embodiments, the nanoparticles have a diameter of around 100 nm. In some embodiments, the nanoparticles have a diameter not exceeding 160 nm. This is advantageous as a diameter of 100-200 nm is considered appropriate for effective targeting. In some embodiments, the nanoparticles fall in a relatively narrow size range, having a polydispersity index of, for example, 0.06 to 0.4.

In some embodiments, preparation of the nanoparticles includes dialysis to remove the organic solvent. Typically, the dialysis membrane has a molecular cutoff of 500-1000 Daltons.

Methods to deliver the formulated nucleic acid into a cell comprise contacting the PLANA with the cell. Some embodiments comprise contacting the PLANA in vitro with cells in culture. Some embodiments comprise contacting the PLANA with cells in vivo. For in vivo use, PLANA can be administered locally or systemically. Depending on the application, the PLANA can be administered topically, orally, nasally, vaginally, rectally, by injection, including intradermal, subcutaneous, intramuscular, intratumoral, intralesional and intravenous injection, or by inhalation. In some embodiments, the PLANA formulation or PLANA formulation nanoparticle comprises one of more nucleic acids. In some embodiments, the cell is contacted with one or more PLANA formulations (in the form of nanoparticles or otherwise), wherein each PLANA formulation comprises a different nucleic acid that the other(s).

In various embodiments, the living cells can be a bacterial cell, a plant cell, an animal cell, and a fungal cell. The bacterial or fungal cell can be a infectious agent present in a multicellular organism, such as a animal or plant. In some embodiments the animal is a human.

Another aspect is a method of treating a disease, comprising contacting a cell from the individual being treated with a PLANA. In some embodiments, the cell in contacted with the PLANA ex vivo. In some embodiments the PLANA is administered to the individual. In some embodiments, the PLANA is in the form of a nanoparticle. In various embodiments, the disease is a cancer, a central nervous system disorder, an autoimmune disease, a genetic disease, a proliferative disease, a hematological disease, an inflammatory disease, a gastrointestinal disease, a liver disease, a lung disease, a kidney disease, a spleen disease, a familial amyloid neuropathy, pain, a metabolic disorder, a psychiatric disorder, a bacterial infection, a viral infection, or a fungal infection.

With respect to any relevant aspect, in some embodiments, that PLANA is used for prophylactic treatment. In some embodiments, it is used as a vaccine. In other embodiments, it is used for therapeutic treatment. In some embodiments, it is used as an antiviral treatment. In some embodiments, it is used as an antibacterial treatment. In some embodiments, it is used to treat a non-infectious disease or disorder, including for example, cancer, an autoimmune disease, a metabolic disease, or a neurodegenerative disease.

In another aspect, the PLANA may be in the form of a composition with a nucleic acid, for example, an siRNA, that may be used to treat or prevent infection, transmission, or acquisition of SARS-CoV-2 (the virus that causes COVID-19) and other coronavirus-related diseases.

Indeed PLANAs can deliver nucleic acids with antiviral activity against a broad range of viruses, in particular enveloped viruses. Examples of suitable DNA viruses include (but are not limited to) Herpesviruses, Poxviruses, Hepadnaviruses, and Asfarviridae. Suitable RNA viruses include (but are not limited to) Flavivirus, Alphavirus, Togavirus, Coronavirus, Hepatitis D, Orthomyxovirus, Paramyxovirus, Rhabdovirus, Bunyavirus, Filovirus, Retroviruses, and Retroviruses.

In another aspect, rather than containing a nucleic acid, a PLANA-like composition contains other negatively charged molecules, and can be used to deliver them into living cells. In some embodiments, the negatively charged molecule is a phosphopeptide or a phosphoprotein. In some embodiments, the PLANA incorporates, and can be used to deliver

A further aspect is a method of delivering a negatively charged molecule into a living cell comprising contacting the cells with a PLANA-like composition. In some embodiments, the negatively charged molecule is a phosphopeptide or a phosphoprotein, or a nucleic acid.

In another aspect, a PLANA or PLANA-like composition comprises one or more nucleic acid, nucleotide, phosphopeptide, phosphoprotein, protein, carbohydrate, lipid, small molecule, food or agriculture product, cosmetic agent, or other commercially available drug. Further embodiments comprise contacting a living cell with the PLANA or PLANA-like composition to deliver the one or more nucleic acid, nucleotide, phosphopeptide, phosphoprotein, protein, carbohydrate, lipid, small molecule, food or agriculture product, cosmetic agent, or other commercially available drug.

In another aspect, the PLANA is used in a cosmetic procedure.

In other aspects, the PLANA is used in agriculture or the food industry.

### Example 1

### Peptide Synthesis

Fmoc solid-phase peptide synthesis was utilized to synthesize the cyclic and linear difatty acyl conjugated peptides, generally as previously described (Do, H., et al., Difatty Acyl-Conjugated Linear and Cyclic Peptides for siRNA Delivery. ACS Omega 2(10):6939-6957, 2017); however, a different strategy was used for the synthesis of CP-C18 using Fmoc-Lys(Dde)-OH for both lysine residues (Hall, R. et al. Peptide/Lipid-Associated Nucleic Acids (PLANAAs) as a Multicomponent siRNA Delivery System. Mol. Pharm. 2021. doi: 10.1021/acs.molpharmaceut.0c00969).

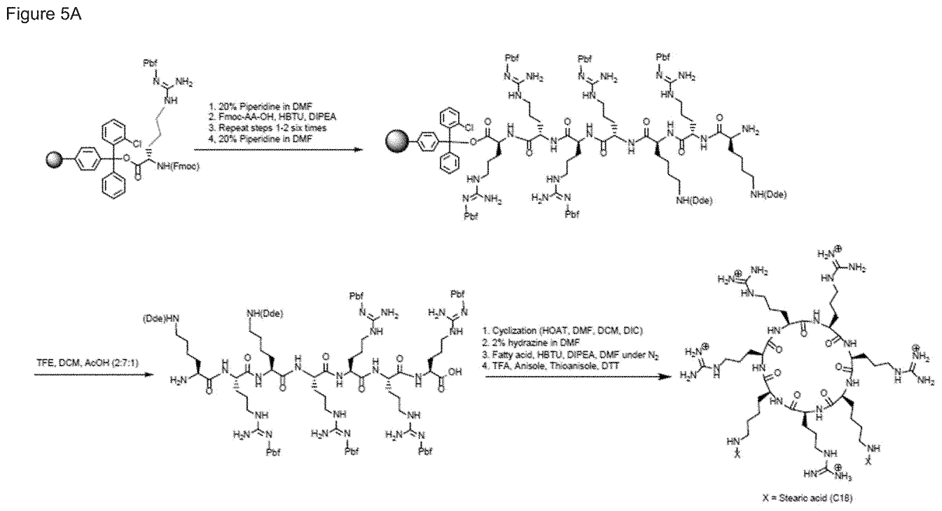

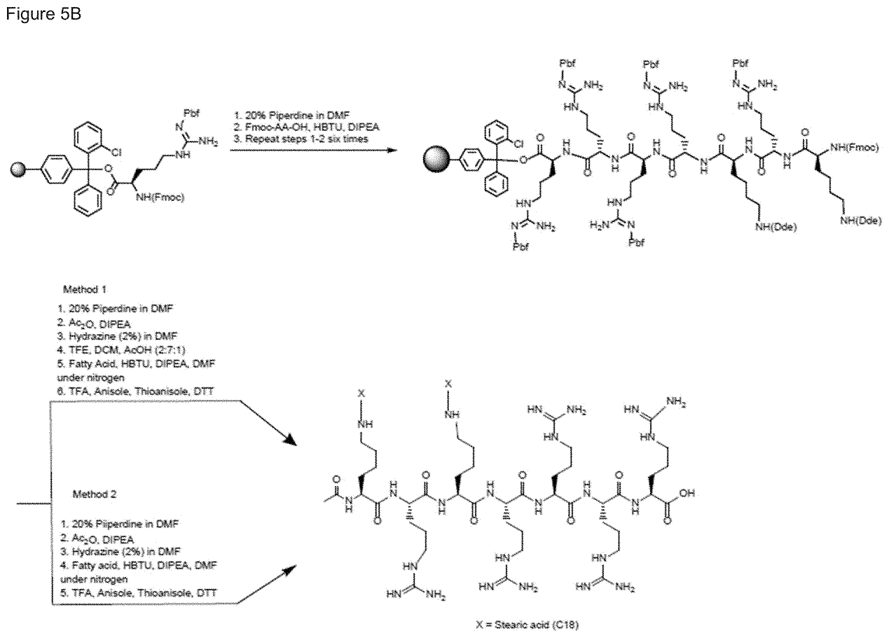

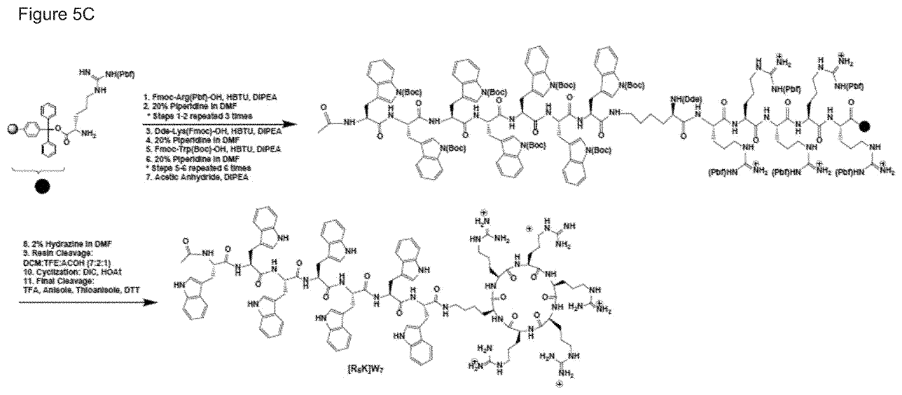

Synthesis of cyclic peptide CP-C18. Briefly, H-Arg(Pbf)-2-CITrt resin (0.40 mmol, 700 mg) was used as the solid support for the synthesis of cyclic peptide [R5K2]. Fmoc-Arg(Pbf)-OH and Fmoc-Lys(Dde)-OH were used as the building block amino acids (FIG. 5A). The resin was swelled in 10 mL of N,N-dimethylformamide (DMF) in a reaction vessel under nitrogen for 30 min. and drained. The coupling of the first amino acid was accomplished by adding 3 equiv. of Fmoc-Arg(Pbf)-OH and 3 equiv. of 2-(1H-benzotriazol-1-yl)-1,1,3,3-tetramethyluronium hexafluorophosphate (HBTU) in 7 mL of DMF and 6 equiv. of N,N-diisopropylethylamine (DIPEA) to the reaction vessel. The reaction was allowed to proceed for 1 h under nitrogen. The mixture was then filtered and washed with 10 mL of DMF for 1 min under nitrogen. The solution was drained and filtered once more. The peptide was deprotected by piperidine in DMF (20% v/v, 10 mL) under nitrogen for 15 min. The solution was filtered, and deprotection was repeated once more. The reaction was then washed for removal of any residual piperidine by 10 mL of DMF under nitrogen for 3 min, and then filtered. The washing was repeated once more. This process was repeated using Fmoc-Arg(Pbf)-OH and Fmoc-Lys(Dde)-OH until the R5K2 peptide chain was assembled on the resin. The cleavage from the 2-chlorotrityl resin was performed using 35 mL dichloromethane (DCM), 10 mL trifluoroethanol, and 5 mL acetic acid for 1.5 h. The solution was then diluted with hexane and evaporated four times. To cyclize the peptide, 3 equiv. of 1-hydroxy-7-azabenzotriazole (HOAT), 200 mL of anhydrous DMF, 40 mL of anhydrous DCM, and 4 equiv. of N,N′-diisopropylcarbodiimide (DIC) were added to the dry peptide under nitrogen. The mixture was allowed to react for 24 h. Once cyclization was complete, 2% hydrazine in DMF was used to remove the Dde protecting groups. Stearic acid was then coupled to the unprotected lysine amino acid residues. This was accomplished by mixing 3 equiv. of the fatty acid with 2.5 equiv. of HBTU and 6 equiv. of DIPEA in DMF. The fatty acid coupling was then repeated to ensure maximum coupling to the unprotected lysine amino acid residues to yield CP-C18. Final deprotection was carried out using 18 mL TFA, 0.50 mL anisole, 0.50 mL thioanisole, and 50 mg DTT for 2.5 h and purified by HPLC as described below. The MALDI-TOF (m/z) data were consistent with expectation.

Synthesis of Linear Peptide LP-C18.

This peptide is different from the previously described peptides since this peptide is capped with an acetyl group. Two strategies were used for the synthesis of LP-C18. H-Arg(Pbf)-2-CITrt resin (0.40 mmol, 700 mg) was used as the solid support for the synthesis of the linear peptides. Fmoc-Arg(Pbf)-OH and Fmoc-Lys(Dde)-OH were used as the building block amino acids (FIG. 5B). The same procedure was used to assemble the linear peptide chain on the resin, as mentioned in cyclic synthesis. Once the peptide sequence was assembled on the resin, the free N-terminus was capped by acetic anhydride (Ac₂O) and HBTU under nitrogen for 1 h. Both Dde protecting groups on the two lysine amino acid residues were then removed by 2% hydrazine in DMF. 15 mL of the 2% hydrazine solution was added to the peptide under nitrogen for 15 min and filtered. This was repeated three more times. The peptides were cleaved from the resin. The fatty acid coupling was performed in the solution phase with stearic acid as previously described, and the peptide was completely deprotected using the same procedure as in the cyclic peptide synthesis (described below) to produce LP-C18.

A second synthesis method was also developed for the linear LP-C18. The same methodology was employed as the previous linear synthesis through Dde removal. Following Dde removal, fatty acid conjugation was conducted with the reaction of 3 equiv. of stearic acid on the peptide-attached resin in the presence of 3 equiv. of HBTU and 6 equiv. of DIPEA under nitrogen for 2 h. Triple coupling was performed to ensure complete fatty acid coupling. The resin was then cleaved using the previously stated procedure.

Synthesis of Hybrid Cyclic-Linear Peptides [R₅K]W₅, [R₅K]W₆, and [R₅K]W₇.

The synthesis of hybrid cyclic-linear peptides, [R₅K]W₅ and [R₅K]W₆ was carried out as previously described (Mozaffari, S., et al., Amphiphilic Peptides for Efficient siRNA Delivery, Polymers (Basel), 11(4), 2019 and Hall, R. et al. Peptide/Lipid-Associated Nucleic Acids (PLANAAs) as a Multicomponent siRNA Delivery System. Mol. Pharm. 2021. doi: 10.1021/acs.molpharmaceut.0c00969). In brief, for the synthesis of [R₅K]W₇, building block amino acids, Fmoc-Arg(Pbf)-OH, Fmoc-Trp(Boc)-OH, and Dde-Lys(Fmoc)-OH were used as building block amino acids in the synthesis. The appropriate resin, H-Arg(Pbf)-2-chlorotrityl (0.44 meq/g, 905 mg), was swelled in DMF under dry nitrogen for 15 min three times. The solvent was filtered off. The next Fmoc-protected amino acid (3 equiv.) was coupled to the N-terminal in the presence of HBTU (3 equiv.) and DIPEA (6 equiv.) in DMF by agitating under dry nitrogen for 1.5 h. The reaction solution was filtered off after the completion of the coupling. The resin was washed with DMF (15 mL, 2×5 min). Fmoc deprotection was performed by using piperidine in DMF (20% v/v, 10 mL, 2×15 min). The reaction solution was filtered off, and the resin was washed with DMF (15 mL, 2×5 min). The subsequent amino acids were coupled and deprotected in a similar manner (FIG. 5C). After conjugation of lysine residue containing the protected amino group with Dde, the Fmoc of the side chain protecting group of lysine was removed in the presence of piperidine in DMF (20% v/v). Coupling of Fmoc-Trp(Boc)-OH on the side chain of lysine was continued. After Fmoc deprotection on the last amino acid, the free amino group was capped in the presence of acetic anhydride (3 equiv.) and DIPEA (6 equiv.). The Dde group of lysine residue was removed in the presence of hydrazine in DMF (2% v/v). The resin was washed with DMF (15 mL, 2×5 min) and methanol (15 mL, 5 min). The resin was dried under vacuum for 4 h. The resin cleavage cocktail, dichloromethane:trifluoroethanol:acetic acid (DCM:TFE:AcOH; 35 mL:10 mL:5 mL) was freshly prepared. The cleavage cocktail was added to the resin, and the solution was mixed for 3 h. The filtrate was evaporated under low pressure. Hexane (2×20 mL) and DCM (2×15 mL) were added to the residue to remove the acetic acid from the mixture. The crude material was solidified as a white solid. The crude peptide was dried under vacuum overnight.

After the formation of the linear peptides was confirmed by MALDI mass spectroscopy, the crude protected peptides were used for the cyclization reaction. Anhydrous DMF (100 mL), anhydrous DCM (50 mL), DIC (188 μL), and HOAt (122.5 mg) were added to the crude unprotected linear peptide for cyclization. The solution was stirred under dry nitrogen overnight. After the formation of a cyclic peptide was confirmed by MALDI analysis, the solvents were removed under reduced pressure. The crude peptide was dried overnight. The cleavage cocktail (TFA, anisole, thioanisole, 9:1:2 v/v/v and 50 mg of DTT, 20 mL total volume) was added to the crude product. The mixture was stirred at room temperature for 4 h. The crude peptide was precipitated in cold diethyl ether and centrifuged, purified by using reversed-phase HPLC, and lyophilized.

MALDI-TOF (m/z): [R₅K]W₇: MALDI-TOF (m/z): C₁₁5H₁₄₉N₃₆O₁₄, calculated: 2253.2027, found: 2254.73 [M+H]+, 2276.75 [M+Na].

### Example 2

### PLANA Formulations

Seven different peptides were initially selected to be incorporated into PLANA formulations from three different categories of peptides: LP-C16 and LP-C18 as linear fatty acyl-conjugated peptides (FIG. 1), CP-C16 and CP-C18 as cyclic fatty acyl-conjugated peptides (FIG. 2), and [R₅K]W₅, [R₆K]W₆, and [R₅K]W₇ as cyclic/linear hybrid peptides (FIG. 3). The C18 peptides generally performed better than the C16 peptides, and only data for the former are described below.

Nanoparticles prepared without incorporation of any of the selected peptides (as a control) were named Lipid-Associate Nucleic Acids (LANA), and were included in the experiments to study the effect of incorporation of peptide into the composition. One LANA and four PLANA formulations were prepared. PLANA formulations were prepared with different compositions by mixing one of the five peptides (LP-C18, CP-C18, [R₅K]W₅, [R₆K]W₆, and [R₅K]W₇), with DOTAP, DOPE, cholesterol, and phosphatidylcholine in one LANA and four PLANA formulations summarized in Table 1, with or without siRNA.

TABLE 1
PLANA compositions (all components are specified
as molar fractions)

| Components | | PLANA | PLANA | PLANA | PLANA |
|---|---|---|---|---|---|
| (Molar fraction) | LANA | A | B | C | D |
| Peptide | — | 0.00625 | 0.0425 | 0.0425 | 0.0425 |
| DOTAP | 0.25 | 0.244 | — | — | — |
| Cholesterol | 0.25 | 0.25 | 0.319 | 0.128 | 0.128 |
| Phosphatidylcholine | 0.25 | 0.25 | 0.319 | 0.510 | 0.256 |
| DOPE | 0.25 | 0.25 | 0.319 | 0.319 | 0.574 |

Due to different concentrations of peptides used in the PLANA A formulation and the difference in the number of cationic amino acids in the selected peptide structures, the nitrogen to phosphate (N/P) ratio (as an important factor in nucleic acid delivery systems) was not the same in different studied formulations. The N/P ratio of the different PLANA A formulations varied in 6.0-7.2 range (Table 2).

TABLE 2
The nitrogen:phosphate (N/P) ratio of different
PLANA formulations

| Peptide/siRNA | PLANA | PLANA | PLANA | PLANA |
|---|---|---|---|---|
| N/P ratio | A | B | C | D |
| LP-C18 | 6.0 | 36.5 | 36.5 | 36.5 |
| CP-C18 | 6.0 | 36.5 | 36.5 | 36.5 |
| [R₅K]W₅ | 6.0 | 35.0 | 35.0 | 35.0 |
| [R₅K]W₇ | 6.0 | 35.0 | 35.0 | 35.0 |
| [R₆K]W₆ | 7.2 | 42.2 | 42.2 | 42.2 |

PLANA Preparation

Aqueous solutions of the peptide and siRNA were prepared, stock solutions of lipids were diluted to the desired concentration in ethanol, and mixed to provide an aqueous:ethanol ratio of 3:1 v/v. PLANAs were formed by passing the mixture through an Avanti Mini Extruder (Alabaster, Ala.) with a 100 nm filter 50 times to obtain nanoparticles around 100 nm. The PLANA formulations were placed in a dialysis bag (Biotech CE dialysis tubing) with a molecular cutoff of 500-1000 Daltons, and diffusion was performed for 10-15 minutes against the purified water as the receiving phase to remove the ethanol. The dialysis time could be extended to remove the traces of ethanol, if required. The efficiency of this approach was evaluated by preparing PLANA A and PLANA C formulations in which the peptide was added to the ethanol solution (instead of aqueous phase).

### Example 3

### Nanoparticle Size and Surface Electrical Charge Measurements

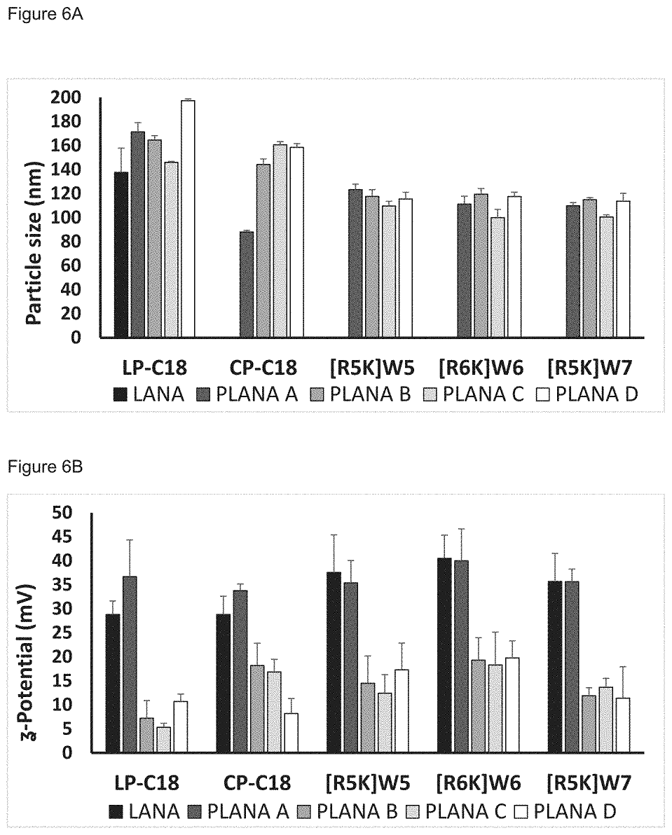

The size and [symbol] -potential of the nanoparticles were measured by dynamic light scattering using a Malvern Nano ZS Zetasizer (Westborough, Mass.). For size measurements, 70 μL of PLANAs was added to a disposable cuvette (Cat. #ZEN0040) at 25° C. For zeta potential measurements, 750 μL of PLANAs was added to a disposable folded capillary cell (Cat. #DTS1070) at 25° C. and surface charge determined at 40 V using Smoluchowski approximation. The size of the PLANAs ranged from 88-197 nm; however, most of formulations demonstrated a particle size of 100-150 nm (FIG. 6A). PLANA formulations incorporating cyclic/linear hybrid peptides generally showed a slightly smaller size; however, a particular trend in the size of the nanoparticles was not observed in comparing different formulations (PLANA A to D) incorporating each peptide. The particle size was relatively uniform with the average polydispersity index (PDI) of 0.135 and ranging from 0.061 to 0.338 (see Table 3). The zeta potential of the PLANAs ranged from 5.3-40.5 mV (FIG. 6B). The zeta potential was generally lower in PLANA formulations B, C, and D, which illustrates that eliminating the cationic lipid (DOTAP) from the PLANA formulation led to a lower zeta potential. A significant difference was not observed in the zeta-potential of the DOTAP-containing formulations for any of the studied peptides. Adding the peptides in the ethanolic phase (instead of the aqueous portion) did not change the size or zeta-potential significantly (data not shown).

TABLE 3
The polydispersity index (PDI) for different
PLANA formulations

| Peptide | Formulation | PDI |
|---|---|---|
| — | LANA | 0.163 |
| LP-C18 | PLANA A | 0.338 |
| | PLANA B | 0.100 |
| | PLANA C | 0.081 |
| | PLANA D | 0.15 |
| CP-C18 | PLANA A | 0.296 |
| | PLANA B | 0.082 |
| | PLANA C | 0.285 |
| | PLANA D | 0.210 |
| [R₅K]W₅ | PLANA A | 0.065 |
| | PLANA B | 0.121 |
| | PLANA C | 0.093 |
| | PLANA D | 0.103 |
| [R₆K]W₆ | PLANA A | 0.122 |
| | PLANA B | 0.088 |
| | PLANA C | 0.090 |
| | PLANA D | 0.100 |
| [R₅K]W₇ | PLANA A | 0.094 |
| | PLANA B | 0.061 |
| | PLANA C | 0.078 |
| | PLANA D | 0.105 |

| | |
|---|---|
| Average | 0.135 |

### Example 4

Electron Microscopy

Scanning electron microscopy (SEM) and transmission electron microscopy (TEM) were used to characterize the nanoparticle formation of PLANAs as well as the peptide alone.

Scanning Electron Microscopy (SEM)

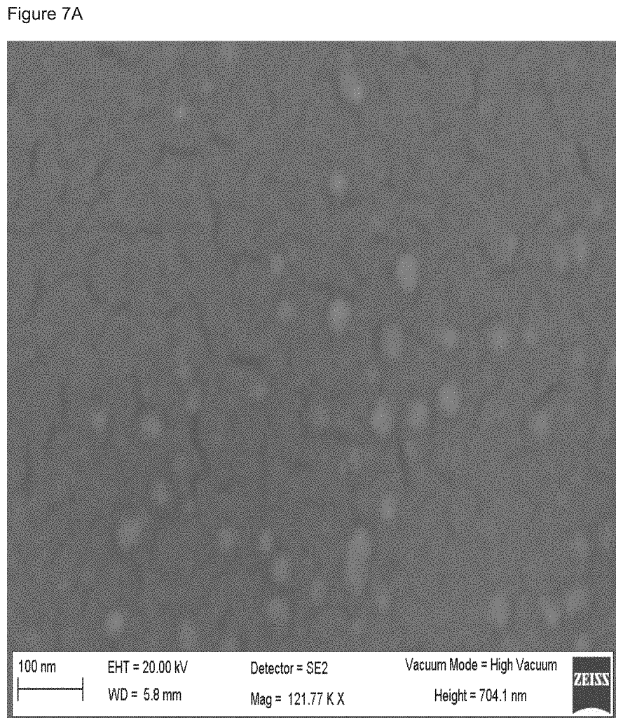

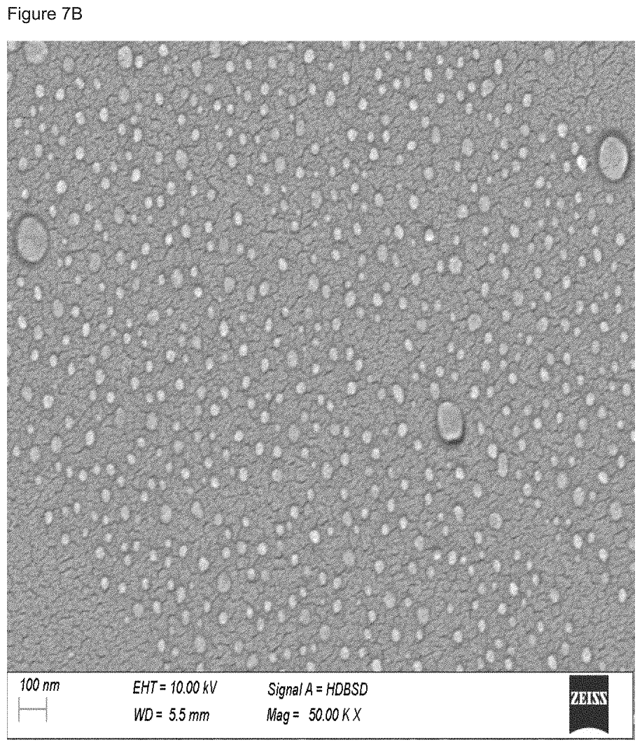

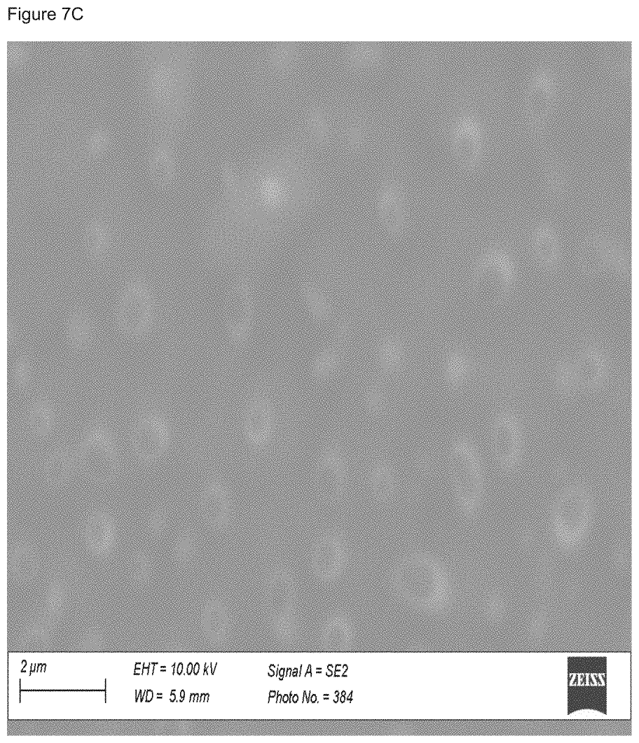

For SEM analysis, LP-C18 and CP-C18 peptides were spotted after 24 h incubation at room temperature to allow peptide self-assembly. LP-C18 PLANAs were formulated and immediately spotted, and allowed to dry before analysis. LP-C18 and LP-C18 PLANAs both show nanoparticle formation around 50-100 nm (FIGS. 7A and 7B), respectively. CP-C18 was found to form bigger nanoparticles, around 500-1000 nm (FIG. 5C). The larger observed nanoparticles suggest that some aggregation may have occurred during the sample preparation after 24 h incubation. Additionally, CP-C18 formed vesicle-like nanoparticles, with a cavity in the middle (FIG. 7C).

Transmission Electron Microscopy

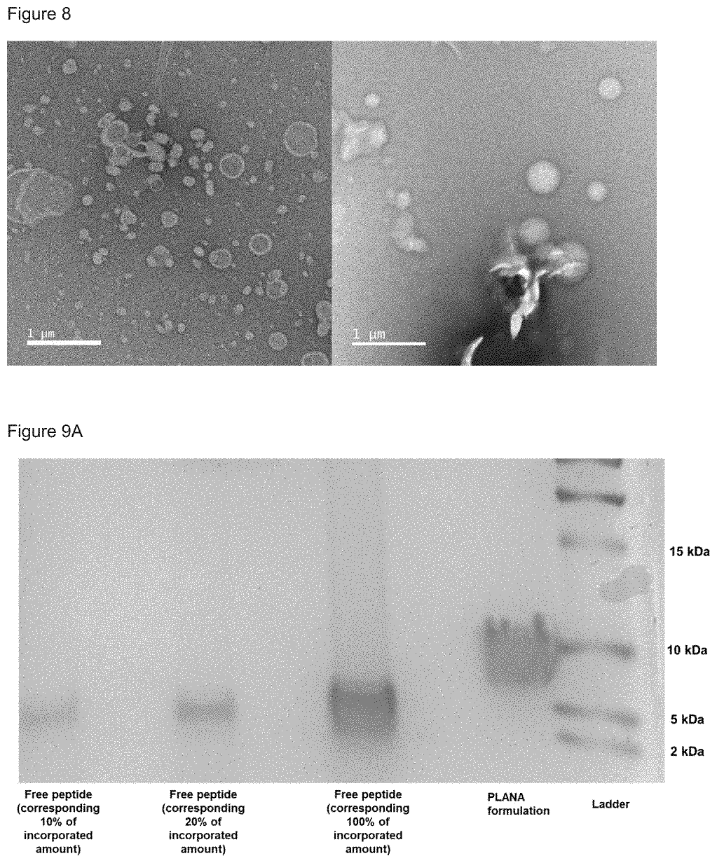

TEM samples were prepared by spotting peptide (5 uL of 0.5 mM solution in H₂O) solution or PLANA onto a carbon-coated copper grid (300 mesh). PLANAs were spotted immediately after formulation preparation. For analysis of the peptide alone, CP-C18 was allowed to self-assemble at room temperature for 24 h and then spotted on a carbon-coated copper grid. The drop was allowed to stay on the coverslip for 10 min. The excess solution was removed from the surface of the grid, and the sample kept overnight to dry. The sample was examined by IMRI using a JEOL JEM-2100 transition electron microscope. Images were recorded on a Gatan Oneview CCD camera at a magnification of 25000× and 10000×. TEM showed that CP-C18 self-assembled to form vesicle-like nanoparticles around 500 nm (FIG. 8, right panel). CP-C18 PLANAs were found to form nanoparticles around 200 nm with some aggregated larger particles (FIG. 8, left panel).

### Example 5

### Confirmation of Peptide Incorporation

Peptide incorporation in the PLANAs was measured using SDS-PAGE gel electrophoresis. Different concentrations of free peptide were compared to PLANAs. 10%, 20%, and 100% of the amount of free peptide in the PLANAs were run parallel to the PLANAs sample. Due to the amphiphilic nature of the peptides in this study, we included a sample PLANA formulation where the peptide was added to the alcoholic portion of the formulation in order to establish any differences in the extent of peptide incorporation due to the method of peptide incorporation. 14 μL of the PLANAs, equivalent to 5 μg of the peptide, was run on a 16% polyacrylamide gel along with different amounts of the free peptide (0.5 μg, 1 μg, and 5 μg). 1× Tris/Tricine/SDS running buffer (Cat. #1610744, Bio-Rad, Hercules, Calif.) was used in a Mini-PROTEAN Tetra Electrophoresis Bio-Rad System. The system was run at 100V and 35 mA until the dye reached the bottom of the gel (approximately 90 min). After electrophoresis was performed, Bio-Safe Coomassie G-250 Stain (Cat. #161-0786, Bio-Rad was used to stain the gel for 1 h and Coomassie Brilliant Blue R-250 Destaining Solution (Cat. #1610438) was used to de-stain the gel for 1 h, changing solution after 30 min. The bands were then visualized using a BioRad Imager (Universal Hood III).

The two methods of peptide incorporation were tested for the PLANA A formulations incorporating LP-C18 peptide, and the movement of the peptide in SDS-PAGE gel in the PLANA formulations was compared to free peptide loaded in amounts equivalent to 10%, 20%, and 100% of the peptide incorporated in the PLANA formulation. The PLANA formulation prepared with the peptide included in the aqueous phase revealed no band corresponding to the free peptide samples (FIG. 9A). However, when the peptide was added to the alcoholic phase, a faint band was observed at the location corresponding to free peptide (FIG. 9B), which indicates a higher efficiency for peptide incorporation into the nanoparticles by mixing the peptide with siRNA in the aqueous phase before exposing it to the lipid mixture in ethanol. We also note that in some of the images, the oligomerization was detected in some of the free peptide bands. [R₅K]W₅ was also evaluated as a representative for the hybrid peptides, and a similar efficiency was noticed, when the peptide was included in the aqueous phase (FIG. 9C).

### Example 6

### Encapsulation Efficiency

For determination of the encapsulation efficiency of the PLANA formulations, a SYBR Green II dye exclusion assay was performed using a wide range of N/P ratios. This method has been extensively used for DNA, and by us for free siRNA quantification. siRNA encapsulation was measured by adding 200 μL of SYBR Green Dye II (Cat. #S7564; 1:10,000 dilution) to 35 μL of the freshly prepared PLANAs sample in a 96 well BD FluoroBlok MW Insert (Cat. #137103). The fluorescence signal was quantified using a SpectraMax M5 V VIZ Plate Reader at a A excitation of 485 nm and a λ emission of 527 nm. Free siRNA in a wide range of concentrations was used to create a standard curve for each set of experiments.

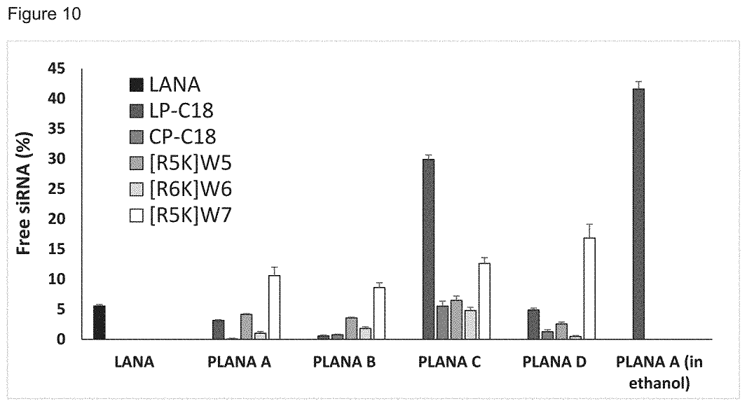

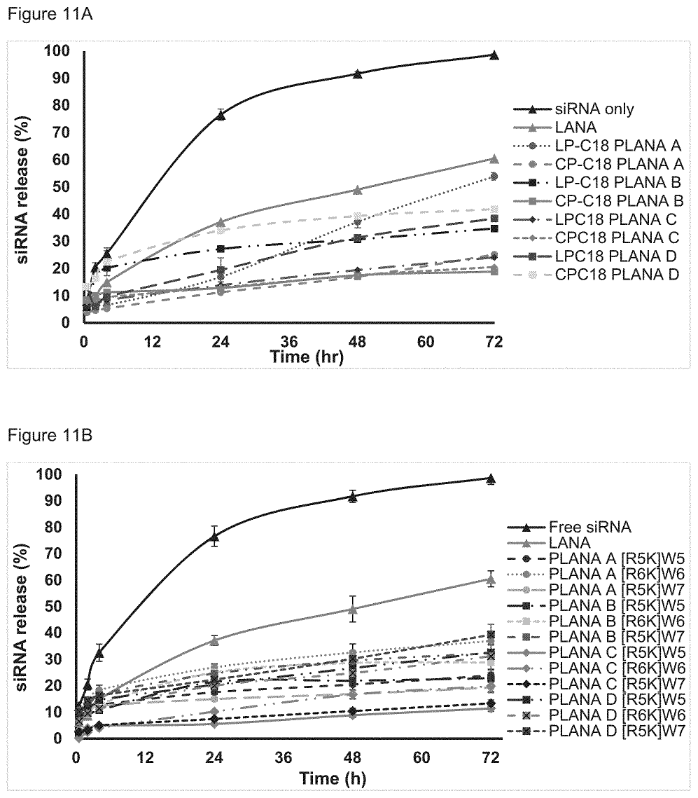

While the LANA formulation (where no peptide was used) showed ˜94.5% encapsulation efficiency (only 5.5% free siRNA was detected), most of peptide-containing formulations demonstrated an equal or higher efficiency. Overall, CP-C18-containing PLANA formulations showed the highest encapsulation efficiency (reaching ˜99.9% efficiency for PLANA A formulation). Among the selected peptides, [R₅K]W₅ showed the lowest efficiency overall, with 9% or more free siRNA detected in different formulations. Addition of peptide in the ethanol phase was again confirmed as a less efficient approach, as the siRNA encapsulation efficiency dropped to ˜58.5%, as compared to ˜96.9% efficiency for the same formulation, when the LP-C18 was included in the aqueous phase (FIG. 10).

### Example 7

### siRNA Release

siRNA release from PLANAs was studied using a dialysis method, and the free siRNA in the receiving phase was quantified using spectro-fluorometry. A standard curve was obtained by preparing different concentrations of siRNA ranging from 30 nM to 0 nM. All samples were added to a 96 well BD FluoroBlok MW Insert. To each sample, 200 μL of SYBR Green Dye II was added, and the fluorescence was read using a SpectraMax M5 UV VIZ Plate Reader. 1 mL of PLANAs were added inside a 20 kDa MW cutoff CE tubing dialysis bag (Cat. #131342) submerged in 30 mL DI water. 250 μL of the receiving phase was sampled at pre-determined time-points (30 min, 2 h, 4 h, 24 h, 48 h, and 72 h). Dialysis bag samples were added to a 96 well BD FluoroBlok MW Insert. To each sample, 200 μL of SYBR Green Dye II was immediately added, and the fluorescent signal was quantified using a SpectraMax M5 UV VIZ Plate Reader as described in Example 6.

Free siRNA was used as a positive control for all PLANA formulations. While ˜77% of free siRNA was detected in the receiving phase after 24 h, near complete diffusion (˜97%) was confirmed at 72 hours' time-point (FIGS. 11A-B). None of the PLANA formulations reached more than 72% release at 72 hours' time-point. The highest release rate was observed for LANA formulation (the only formulation that did not include any of the peptides in the composition), which showed 37 and 72% release at 24- and 72-hours' time-points, respectively.

Among PLANA formulations prepared with fatty acyl-conjugated peptides, no specific trend was observed; however, PLANA formulations A and B incorporating LP-C18 showed a higher release at the endpoint of the experiment (72 h) compared to corresponding formulation incorporating CP-C18 (PLANA A formulation: 54% for LP-C18 vs. 25% for CP-C18; PLANA B: 37% for LP-C18 vs. ˜19% for CP-C18). For PLANAs C and D, the values were comparable at 72 hours' time-point for these two peptides (FIG. 11A). For PLANA formulations prepared with hybrid peptides, PLANA C formulations generally showed some of the slowest release rates, with ˜11%, 13%, and ˜20% release at 72 hours' time-point for [R₅K]W₅, [R₅K]W₇, and [R₆K]W₆, respectively (FIG. 11B). On the other hand, PLANA D formulations showed a higher release at the endpoint compared to other hybrid peptide-containing PLANA formulations (31%, 32%, and 39% release at 72 hours for [R₆K]W₆, [R₅K]W₅, and [R₅K]W₇, respectively). No specific trend was noticed for PLANA A and B formulations for hybrid peptides.

A mathematical analysis of the siRNA release rate data using the one-way association model and equation:

Y=Y0+(Plateau−Y0)×(1−exp(−KX),

where Y is the percentage released, and the Y₀ is the Y value when X (time) is zero. It is expressed in the same units as Y. Plateau is the Y value at infinite times, expressed in the same units as Y. K is the rate constant, expressed in min⁻¹. The R² values were in the range of 0.94-0.99 for all the formulations. The K values are shown in Table 4. The rate of siRNA release slowed down for all PLANA formulations when compared with free siRNA. However, the presence of specific peptides and the type of PLANA formulation affected the siRNA release significantly. PLANA C formulation with [R₅K]W₅, PLANA D formulation with [R₅K]W₇, PLANA A and PLANA D with LP-C18, and PLANA A with CP-C18 exhibited a significantly lower rate of release when compared with siRNA, LANA, and other formulations.

TABLE 4
The K values calculated for the siRNA release
profile for different PLANA formulations

| Formulation | K (min⁻¹) |
|---|---|
| Free siRNA | 0.05934 |
| LANA | 0.02778 |
| PLANA A [R₅K]W₅ | 0.01974 |
| PLANA A [R₆K]W₆ | 0.03642 |
| PLANA A [R₅K]W₇ | 0.03166 |
| PLANA B [R₅K]W₅ | 0.0812 |
| PLANA B [R₆K]W₆ | 0.07648 |
| PLANA B [R₅K]W₇ | 0.06523 |
| PLANA C [R₅K]W₅ | 0.000021 |
| PLANA C [R₆K]W₆ | 0.02162 |
| PLANA C [R₅K]W₇ | 0.01207 |
| PLANA D [R₅K]W₅ | 0.0172 |
| PLANA D [R₆K]W₆ | 0.02121 |
| PLANA D [R₅K]W₇ | 0.003171 |
| PLANA A LP-C18 | 2.11E−06 |
| PLANA A CP-C18 | 4.11E−06 |
| PLANA B LP-C18 | 0.2721 |
| PLANA B CP-C18 | 0.01417 |
| PLANA C LPC18 | 0.01149 |
| PLANA C CPC18 | 0.01115 |
| PLANA D LPC18 | 0.005452 |
| PLANA D CPC18 | 0.06249 |

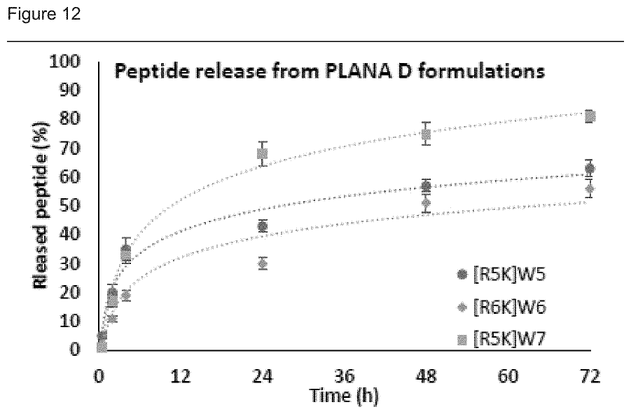

The release profile for the hybrid peptides was also examined in a similar experiment using PLANA D formulations incorporating [R₅K]W₅, [R₆K]W₆, and [R₅K]W₇. The [R₅K]W₇ was released significantly faster and to a higher end-point extent (˜81% after 72 hours) compared to the other two selected peptides. [R₆K]W₆ showed the most extended release profile (˜55% after 72 hours) among the three (FIG. 12).

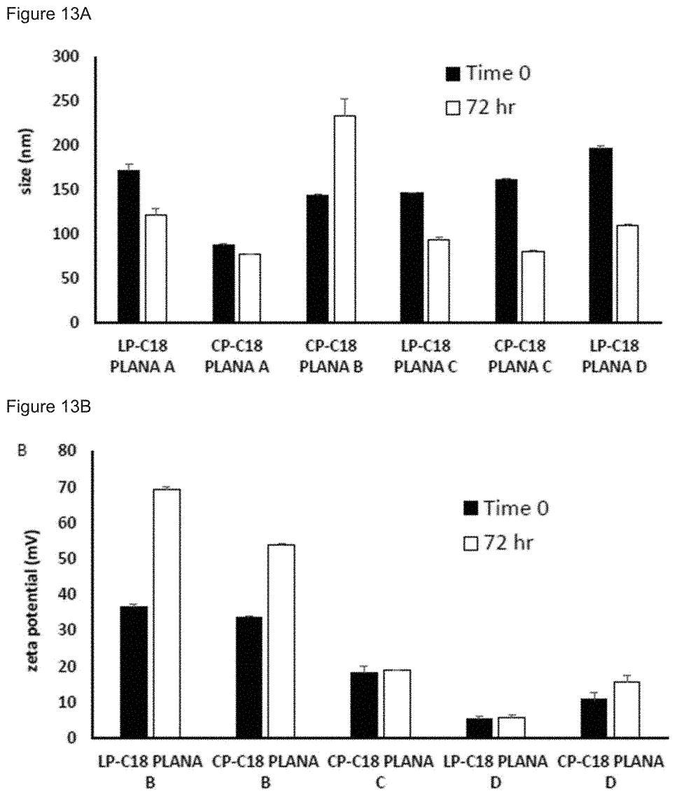

The size and [symbol] -potential of selected PLANA formulations prepared with fatty acyl-conjugated peptides was measured and compared before the dialysis experiments and after 72 h of release test to evaluate potential changes (FIG. 13). The size of the PLANA formulations before and after the release study indicated that most of the selected formulations decreased in size after the 72-h dialysis experiment. The only exception was CP-C18-containing PLANA B formulation, where the average size was increased to 233 nm (compared to 144 nm at time zero; FIG. 13A). Electrical surface charge values showed a significant increase in the [symbol] -potential for DOTAP-containing PLANA formulations (as a positively charged lipid) at 72-hour time-point. Such drastic change was not observed for other selected formulations (FIG. 13B).

### Example 8

### Cytotoxicity

A critical characteristic of an ideal delivery system is its safety profile. Cytotoxicity was evaluated by exposing MDA-MB-231 cells (a triple-negative breast cancer cell line) to various PLANA formulations.

Human breast cancer cell line MDA-MB-231 was obtained from American Type Culture Collection (ATCC; Manassas, Va.; Cat. #: HTB-26™). The cell line was cultured in Dulbecco's modified Eagle's medium supplemented with 10% (v/v) FBS, 100 μg/mL streptomycin, and 100 U/mL penicillin. The cells were maintained at 37° C. and 5% CO₂ and subcultured when 80-90% confluent. Cells were discarded after 30 subcultures, and a new batch of cells was thawed and cultured according to the same protocol. Human MRC-5 lung fibroblast cells (ATCC® CCL-171™), H9C2 human myocardium cells (ATCC® CRL-1446™), PSC human epidermal keratinocytes (HEKa) (ATCC® PCS-200-011™), and HepaRG™ human hepatocytes (ThermoFisher Scientific; HPRGC10) were also included in the cytotoxicity of the PLANAs, and were grown according to the manufacturer's guidelines.

MDA-MB-231 cells were seeded in a 96-well plate. Once 70% confluency was reached, the cells were treated in triplicate with one of the following groups: normal saline (no treatment; NT), PLANAs equivalent to 25, 50, 75, or 100 nM siRNA, or similar concentrations of PLANA formulations without siRNA. PLANAs with LP-C18 and CP-C18 peptides were studied in this set of experiments. Cells were exposed to the different reagents and were incubated at 37° C. and 5% CO₂ for 48 h. A Cell Counting 8 (CCK8) KIT (Biotool; Houston, Tex.; also known as WST-8) was used to evaluate the potential toxicity of the PLANA formulations on the viability of the cells. After the incubation period, 10 μL of CCK solution (Cat. # B34304) was added to each well, and plates were incubated at 37° C. for 1-2 h. The absorbance was then measured at 450 nm using a SpectraMax M5 UV VIZ Plate Reader to determine cytotoxicity as a percentage of viable cells compared to the no treatment cells (after eliminating the signal from “Blank” wells (cell-less medium with CCK-8 solution added). All study groups were evaluated in triplicates.

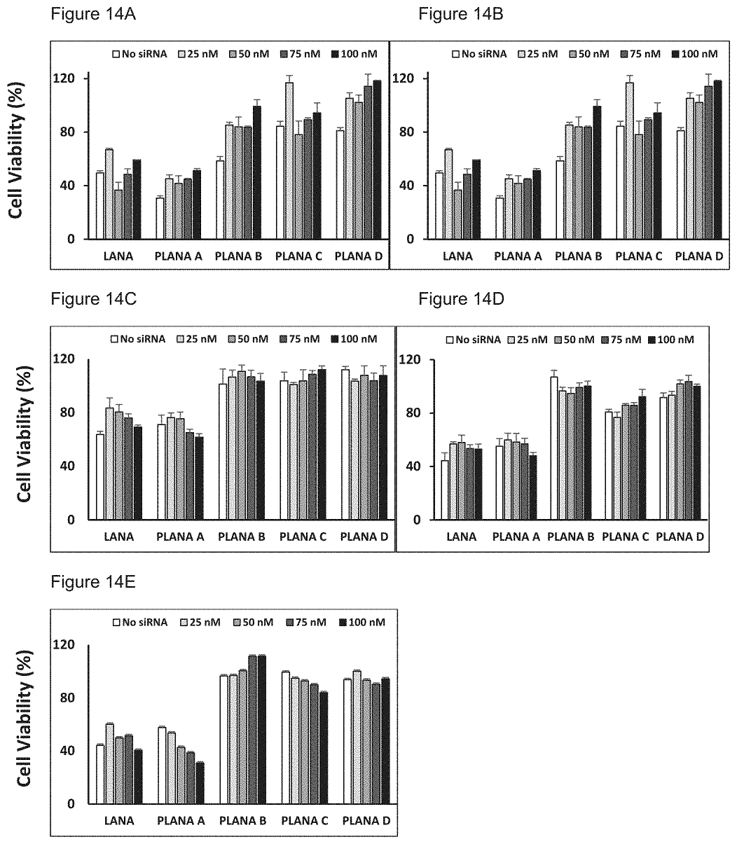

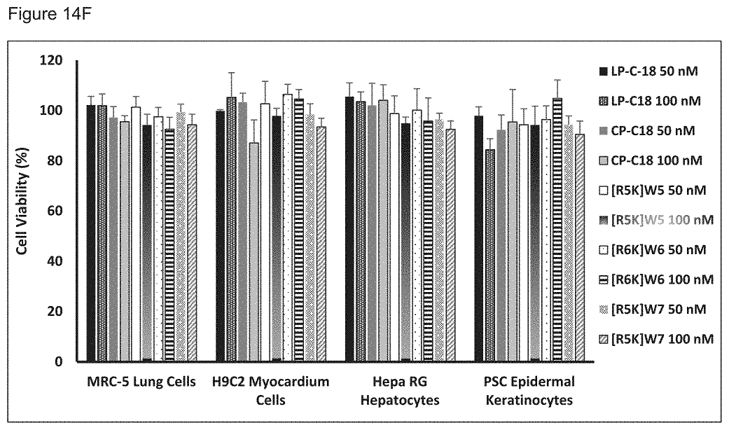

Formulations LANA and PLANA A (the only PLANA formulations containing DOTAP, as a cationic lipid) were found to be toxic to the MDA-MB-231 cells for all selected peptides (FIGS. 14A-E). The highest drop in cell viability was observed in [R₅K]W₇-containing PLANA A formulation at a concentration equivalent to 100 nM siRNA (˜31% viability). No significant difference was observed between LANA and PLANA A formulations for any of the peptides. No significant toxicity was observed for the other three formulations, where blank (no siRNA) LP-C18-containing PLANA D formulation showed the lowest viability among all study groups (˜81% A viability). To further ensure the safety of the PLANA formulations not containing DOTAP, the cytotoxicity of PLANA D formulation was also evaluated in four non-cancerous healthy human cell lines: MRC-5 human lung cell line, H9C2 human myocardium cell line, HepaRG™ human hepatocytes, and PSC human epidermal keratinocytes (HEKa) (FIG. 14F). No significant toxicity was observed for any of the selected PLANA formulations, even at the highest concentration included in the study.

### Example 9

### Cellular Internalization

Flow cytometry. MDA-MB-231 cells were treated with one of the following: Normal saline (no treatment; NT), AF488-siRNA only, AF488-siRNA/peptide complex, AF488-siRNA/peptide/DOPE complex, one of the five different PLANA formulations, or Lipofectamine® 2000 (as positive control) encapsulating AF488-labeled siRNA. (AF488-labeled siRNA was AllStars negative control siRNA labeled with Alexa Fluor 488; Catalogue number 1027292) Experiments were performed with two peptides: LP-C18 and CP-C18. All treatment groups had a final siRNA concentration of 36 nM. The cells were seeded in a 24-well plate, and once 70% confluency was reached, study reagents were added to the cell culture in triplicate wells. Lipofectamine® was used according to the manufacturer's guidelines. After 24 h incubation at 37° C. and 5% CO₂, the media was removed, and the cells were detached using 0.05% trypsin. The cells were fixed using 3.7% formaldehyde in 1×PBS and transferred to 3 mL FACS tubes. Each sample was evaluated using FACSVERSE flow cytometer (BD Biosciences; San Jose, Calif.). The fluorescein isothiocyanate (FITC) channel was used to quantify cell-associated fluorescence. Following each flow cytometry analysis, the percentage of cells positive for fluorescence signal and the mean fluorescence of the cell population was calculated using the calibration of the signal gated with NT cells in order to obtain an autofluorescence of approximately 1% of the population in “no treatment” group.

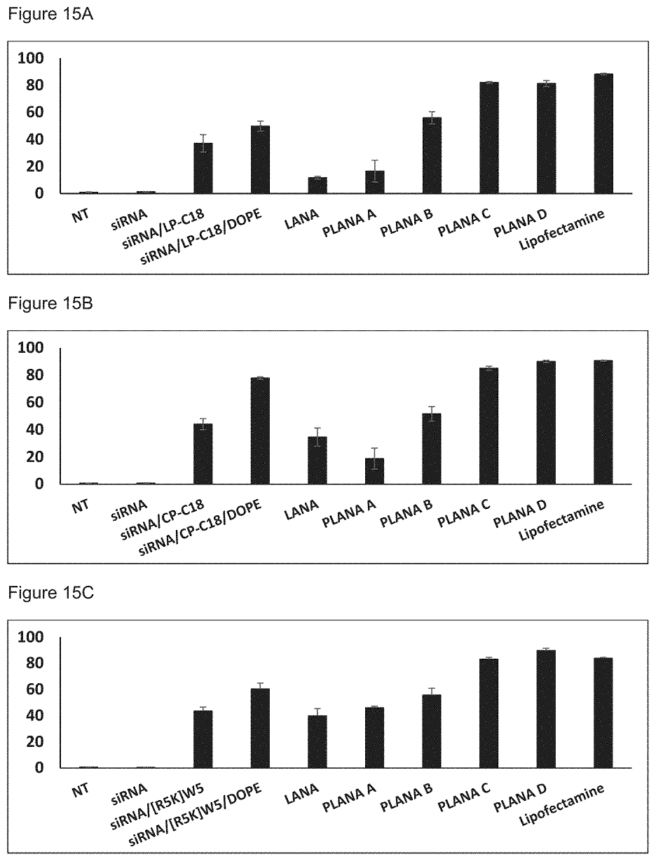

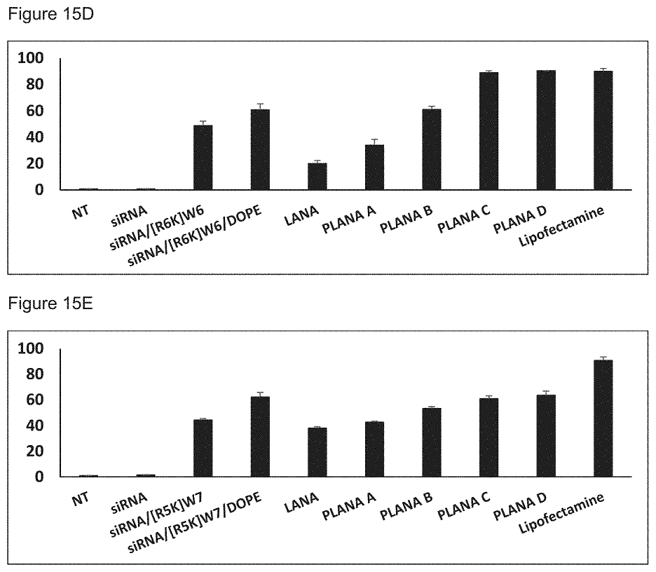

Flow cytometry was performed to quantify the cellular internalization of siRNA in the MDA-MB-231 cells using Alexa Flour 488-labeled siRNA. The study groups for each selected peptide were: Saline (No Treatment, or NT), free siRNA, siRNA/peptide complex, siRNA/peptide/DOPE complex, LANA, PLANA A, PLANA B, PLANA C, PLANA D, and Lipofectamine®/siRNA complexes. The final siRNA concentration was 36 nM for all study groups (except NT), and the percentage of cells positive for siRNA signal is summarized in FIG. 15A-E. For LP-C18, CP-C18, [R5K]W5, and [R6K]W6, PLANA formulations C and D provided the highest uptake (˜82-88% and 81-90%, respectively), which was comparable to Lipofectamine® internalization efficiency. While in most cases PLANA D performed slightly better, no significant difference was observed in the efficacy of these two formulations for these peptides. PLANA B formulations had higher efficiency than LANA and PLANA A formulations (DOPE-containing formulations and performed comparably to siRNA/peptide/DOPE in most cases.

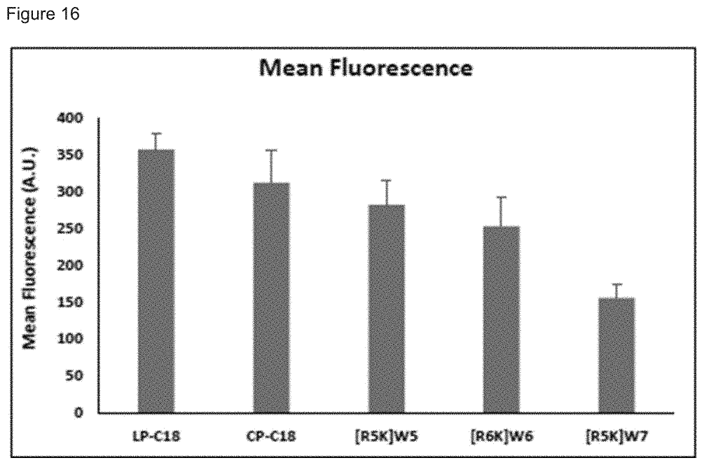

PLANA formulations containing [R₅K]W₇ did not follow this trend, and while PLANA formulations B, C, and D (˜53, 61, and 63%, respectively) showed a higher internalization efficiency than LANA and PLANA A (˜38 and 43%), there was no improvement in siRNA cellular internalization compared to siRNA/peptide/DOPE complex (˜62%) performance. The addition of DOPE to peptide/siRNA complexes improved the internalization efficiency significantly for all selected peptides. Mean fluorescence (MF) of PLANA D formulations prepared with the peptides selected for this project is summarized in FIG. 16. LP-C18 showed the highest mean fluorescence values among the formulations; however, the MF value was not significantly higher than CP-C18. PLANAs incorporating hybrid peptides showed a lower mean fluorescence in comparison.

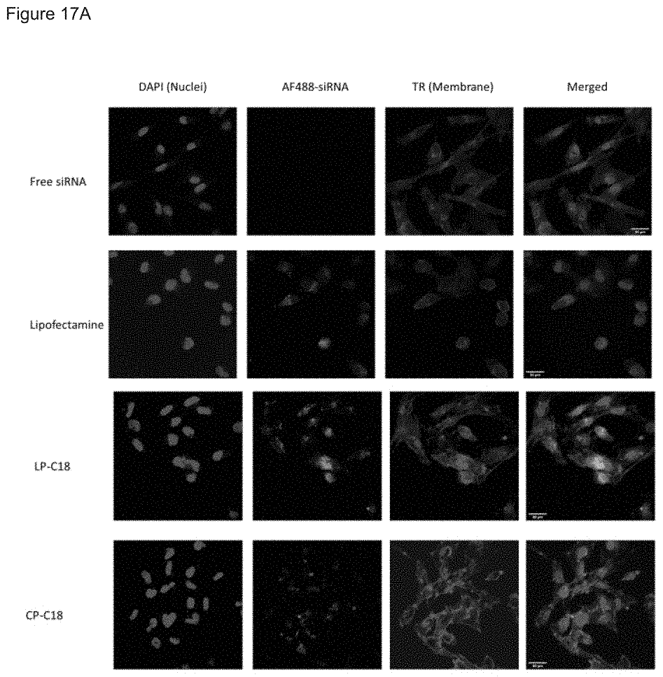

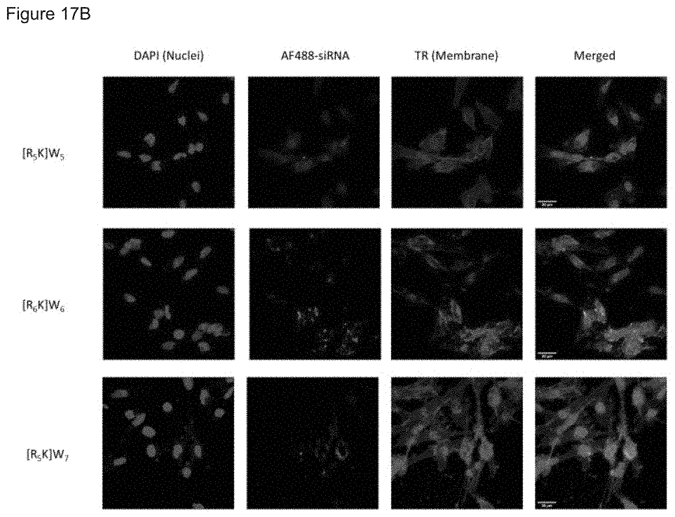

Confocal Microscopy. Additional uptake studies were performed using PLANA D formulations and confocal microscopy to corroborate the results obtained from flow cytometry and to locate and visualize the siRNA inside the cells (FIGS. 17A-B). MDA-MB-231 cells were treated with one of the following: AF488-siRNA only, PLANA D formulations with all five selected peptides (LP-C18, CP-C18, [R₆K]W₆, [R₅K]W₅, and [R₅K]W₇) encapsulating AF488-labeled siRNA or and Lipofectamine®/siRNA. Sterilized cover slips were placed at the bottom of wells in 6-well plates and were exposed to 10% FBS in DMEM and were incubated for 30 min at 37° C. to enhance the cell adherence to the cover slip surface. After incubation, the FBS solution was removed, and cells were seeded on top of the cover slips. Once 70% confluency was reached the study groups were added. After 24 h incubation at 37° C. and 5% CO₂, the media was removed, and cells were washed three times with 1×PBS. Fixing solution (3.7% formaldehyde in 1×PBS) was added for 10 min. The cells were rinsed 3 times in 1×PBS for 5 min. To stain the cell membrane, Texas Red Phalloidin solution (40 μL and 10 mg BSA in 10 mL of 1×PBS) was added to the cells and incubated at room temperature for 1 h. The cells were then washed 3 times with 1×PBS for 5 min. One drop of DAPI was added to a slide to stain the nucleus, and the coverslips were placed face down without air bubbles and were stored overnight away from light to dry. Once dry, a Nikon A1R high definition resonant scanning confocal microscope and a NIS-Elements software (AR 4.30.02, 64 bit) were used to image the cells.

The microscopic images are presented in FIGS. 17A-B. The study group with siRNA only showed no uptake, a finding that was expected as siRNA is unable to internalize into cells without a delivery system. All PLANA D formulations demonstrated effective siRNA internalization in the selected cell line as compared to Lipofectamine®.

### Example 10

### Protein Silencing

siRNA delivery. RPS6KA5 (also known as MSK1) was selected as the model protein for silencing efficiency studies. MDA-MB-231 cells were seeded in 6-well plates as was described in previous sections. After reaching 50% confluency, cells were treated with one of the following groups: no treatment, siRNA targeting RPS6KA5 delivered by Lipofectamine® 2000, or PLANA D formulations. Lipofectamine® was used according to manufacturer's guidelines as was explained in the previous section. The final siRNA concentration in the wells was 100 nM. The cells were incubated at 37° C. and 5% CO₂ for 48 h.

Protein extraction. After 48 h of exposure to silencing siRNAs, media were removed, and the cells were washed with HBSS. In order to detach the cells, 400 uL of 0.05% trypsin was added to each well and plates were incubated at 37° C. for 2 min. The trypsin was deactivated with 2 mL DMEM medium in each well. The cells for each treatment group were transferred to a 15 mL conical tube and centrifuged at 600 rpm for 5 min. The supernatant was discarded, and 1 mL 1×PBS was added to the pellet. The pellet was resuspended by pipetting up and down, and the cells were transferred to a 1.5 mL Eppendorf tube. The cells were centrifuged at 3400 rpm for 5 min. The supernatant was discarded and 35 μL of 1×Ripa buffer (containing protease and phosphatase inhibitors) was added to the pellet and pipetted up and down. All tubes were kept on ice for 5 min, and then sonicated for 2 min. The tubes were vortexed and kept on ice for 1 h, while vortexed every 15 min. The tubes were then centrifuged at the highest speed for 15 min at 4° C. The supernatant, containing protein, was collected in a new 1.5 mL Eppendorf tube and the pellet was discarded.

Protein quantification. To quantify the total protein concentration, a portion of extracted protein was diluted 10 times in 1×PBS in a clean 1.5 mL Eppendorf tube. In a 96-well plate, 25 μL of each sample was added in duplicates. Standards were prepared according to manufacturer's instructions and 25 μL of each standard concentration was added to the same 96-well plate in triplicate. 200 μL of reagents A and B were added to each well, and the plate was placed on a shaker for 30 sec. The plate was then centrifuged at 1500 rpm for 2 min then incubated at 37° C. for 30 min. The samples were then analyzed using a spectrophotometer set at 562 nm.

Western blot. For each study group, 10 μg of total extracted protein (volume calculated based on the protein quantification) was added to a 1.5 mL Eppendorf tube with enough 1×PBS to reach 25 μL final volume. To each sample, 25 μL dye was added, and tubes were vortexed and centrifuged at the highest speed for 1 min. Tubes were then incubated at 95° C. for 5 min. The samples were centrifuged again and put on ice. The samples were loaded on a Mini-Protean TGX 10% gel and run at 100 V and 75 mA for approximately 1 h. The protein was then transferred to a membrane using a BioRad TransBlot Turbo Transfer System at 25 V and 1 A for 45 min. Once the proteins were transferred, the membrane was washed in 1×TBS for 5 min. Then blocking was performed by adding 1 g BSA in 20 mL TBST to the membrane for 1 h at room temperature. The primary antibody (1:1000) in 3% BSA in TBST was then added and left overnight at 4° C. The following day, the membrane was washed with TBS-T six times (5 min each time) and was subsequently incubated with corresponding secondary antibody in 3% BSA in TBST (1:1000) for 1 h at room temperature. Then, the membrane was washed for 5 min four times in TBST. The blots were detected by ECL Detect Kit using ChemiDoc imager (Bio-Rad). The same procedure was followed for visualizing GAPDH as the endogenous gene using corresponding antibodies to normalize the intensity of the bands of the targeted protein.

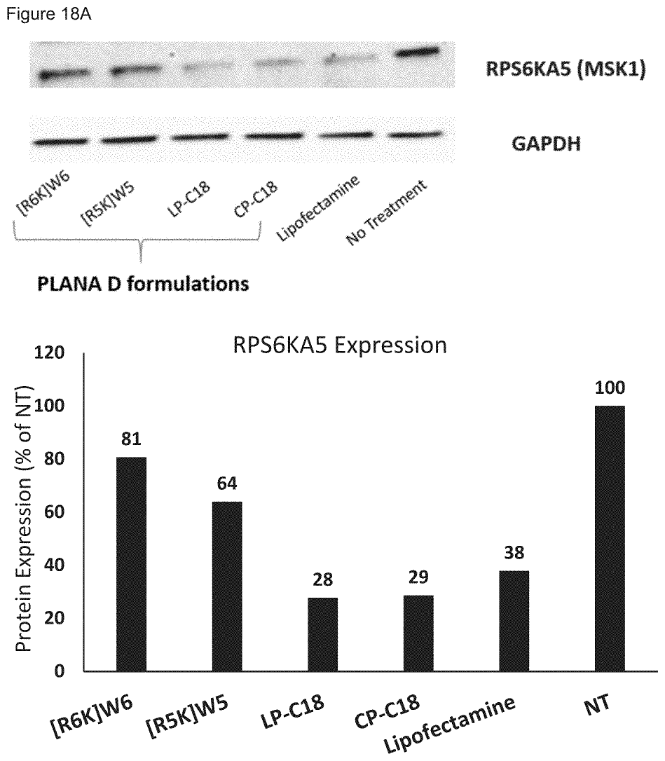

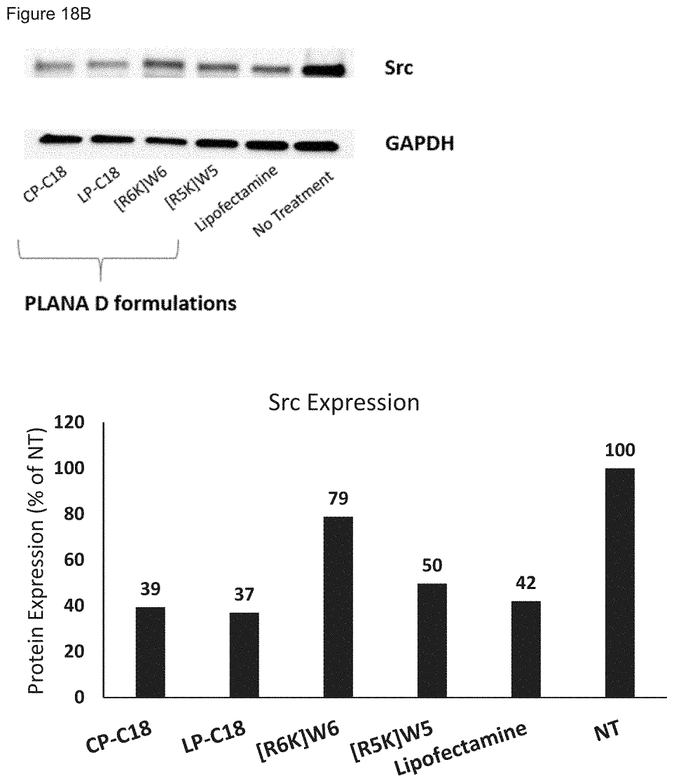

Protein silencing efficiency was evaluated in MB-231 cell line, after targeting the proto-oncogene tyrosine-protein kinase Src and kinase RPS6KA5 (MSK1) proteins. Western blot was performed with Lipofectamine® delivered siRNA as a positive control and saline as the negative control. To normalize the intensity of the bands of the targeted protein, the expression of GAPDH was also evaluated in all study groups as the endogenous protein. Effective silencing was observed for all PLANA D formulations, and the silencing efficiency was similar to (or slightly stronger than) Lipofectamine® for LP-C18 and CP-C18 for both selected targets (28 and 29% of expression by the saline treated cells, respectively, for RPS6KA5 silencing as compared to 38% for Lipofectamine®; 37 and 39% expression, respectively, for Src silencing as compared to 42% for Lipofectamine®; FIGS. 18A and 18B). While PLANA D formulations incorporating [R₅K]W₅ and [R₆K]W₆ showed some level of silencing, the efficiency was lower than PLANAs incorporating fatty acyl-conjugated peptides or Lipofectamine®; however, formulations showed a higher efficiency in silencing Src as compared to RPS6KA5 (50 and 79% expression, respectively, for Src silencing as compared to 64 and 81% expression, respectively, for RPS6KA5 silencing).

### Example 11

### Inhibition of Coronavirus Infection

The viability of viral transduced cells and plaque formation was assessed to determine the effect of siRNA delivery in inhibiting viral infection in human MRC-5 lung fibroblast cells (ATCC® CCL-171™). The viability of healthy and hCoV-229E-infected MRC-5 cells was evaluated using the MTT assay after siRNA delivery targeting the coronavirus S-protein, E-protein, or a combination of both (each representing half of the concentration compared to individual siRNA groups) in a variety of concentrations (ranging from 25 to 100 nM of total siRNA). The untreated MRC-5 cells were included to confirm the safety profile observed in cytotoxicity studies when siRNAs were delivered to the cells. Remdesivir was used as the positive control in this set of studies.

The antiviral studies were performed to test the efficacy of targeting spike and envelope proteins individually and simultaneously, to investigate the potential benefit of combinatorial silencing of two viral targets via delivering a cocktail of siRNAs. For this set of studies, remdesivir was used as a positive control, and media only was used as a negative control. Polymer/siRNA complexes or PLANA were prepared to deliver 100 nM of siRNA targeting mRNA sequence for spike protein expression, 100 nM of siRNA targeting mRNA sequence for envelope protein, or a combination of 50 nM of each siRNA. Treatments were added to the plate at 1 h prior to infection. The polymers used for siRNA delivery were LeuFect B (batch numbers 12-18-6A and 12-18-9A) from RJH Biosciences (Edmonton, Canada). The PLANA formulation used LP-C18 as the peptide component and PLANA D as the lipid component. P3 virus stock was added at 2 TCID₅₀ units per well in a total of 100 μl of serum-free DMEM and incubated at 37° C. for one h. After the initial incubation, an additional 100 μl of DMEM containing 4% FBS was added to each well to make a final concentration of 2% FBS and incubated at 37° C., 5% CO₂. At 7 days post-infection, cell viability was assessed using MTT assay (Promega) according to manufacturer instructions. Briefly, media was removed and discarded to leave 100 μl in each experimental well. MTT reagents were mixed and added directly to cultures (20 μl/well) and allowed to incubate for 1 h at 37° C. 50 μL of 10% SDS was then added to stop the MTT reaction and inactivate the virus for analysis. Inactivation was allowed to proceed for 18 h at room temperature. After inactivation, the MTT colorimetric signal was analyzed using a Spectramax M5 plate reader.

The siRNAs targeting spike (S) and envelope (E) proteins have the following sequences:

Spike protein:
(SEQ ID NO:1)
Forward - 5'-Guu AAA UUU UUU UCG-3'
(SEQ ID NO: 2)
Reverse - 5'- CGA AAA ACA UAC ACU GCC AAA UUU AAC-3'
Envelope protein:
(SEQ ID NO: 3)

| | |
|---|---|
| Froward - 5'- GUU AAA UUU GGC AGU | GUA UGU UUU UCG-3' |

(SEQ ID NO: 4)

| | |
|---|---|
| Reverse - 5'- CGA AAA ACA UAC ACU | GCC AAA UUU AAC-3' |

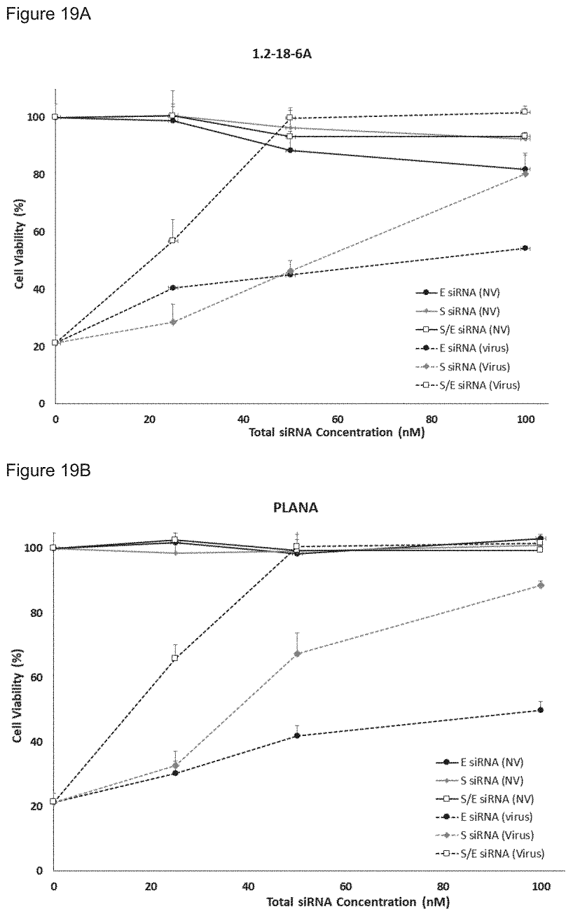

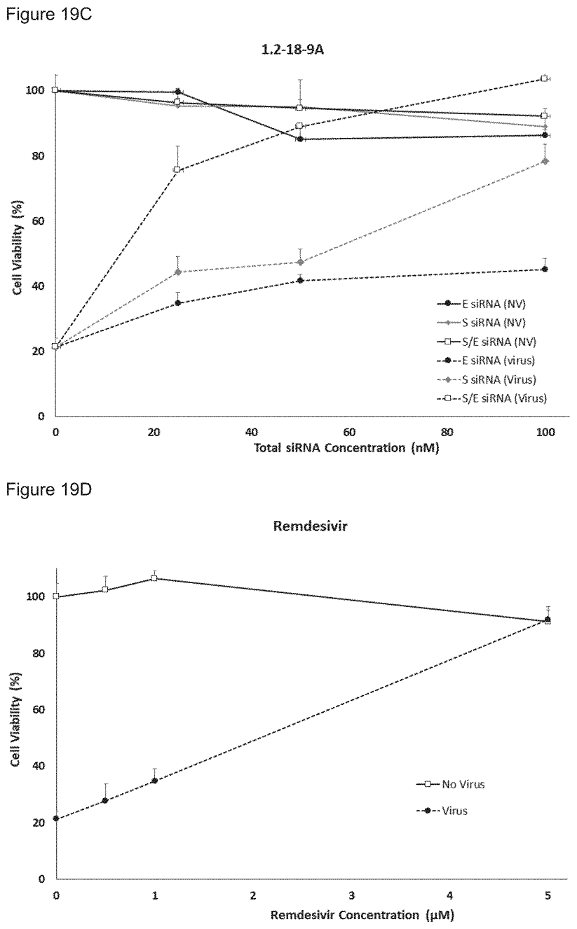

The siRNA delivery (in the absence of viral infection) did not affect the cell viability significantly, which again confirmed the minimal cytotoxicity of the siRNA treatments on the lung cells. The lowest viability observed in the normal MRC-5 cells was in the cells exposed to E-protein siRNA lipopolyplex (˜81 and ˜89% viability for 100 nM of siRNA delivered by 1.2-18-6A and 1.2-18-9A, respectively; FIGS. 19A and 19C). Infecting MRC-5 cells with hCoV-229E decreased the viability to ˜21%, clearly indicating the cytopathic effect of hCoV-229E infection on MRC-5 cells. In hCoV-229E-infected cells, delivering S-protein targeting siRNA preserved cell viability to 80, 79, and 88% with 100 nM siRNA delivered by 12-18-6A, 12-18-9A, and PLANA (FIG. 19B), respectively. Targeting E-protein was less effective in preserving cell viability for all selected delivery systems (54, 45, and 50% cell viability with 100 nM siRNA delivered by 12-18-6A, 12-18-9A, and PLANA, respectively). Silencing S-protein and E-protein simultaneously (50 nM for each siRNA) completely restored cell viability for all three delivery systems. When at 50 nM siRNA combinations (25 nM of each siRNA), cell viability was increased to 90-100% for 12-18-6A, 12-18-9A, and PLANA. Treating the hCoV-229E-infected MRC-5 cells with 5 μM remdesivir restored the viability to 92% (FIG. 19D).

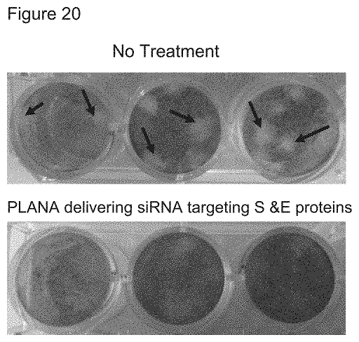

Inhibition of hCoV-229E infection was also assess by plaque reduction assay. MRC-5 cells were seeded in 24-well plates at 1×10⁵ cells per well 24 h before the experiment. Polymer/siRNA complexes or PLANA were prepared to deliver 100 nM of siRNA targeting mRNA sequence for spike protein expression, 100 nM of siRNA targeting mRNA sequence for envelope protein, or a combination of 50 nM of each siRNA and were added to the wells one hour prior to infection. 5-fold dilutions of p3 229E virus stock in 500 μl of serum-free DMEM were then added in triplicate such that, for each treatment condition, 3 wells were infected at TCID₅₀ 8, 1.6, 0.32, and Mock (no virus). After one hour of infection at 37° C., overlay media containing 1% agarose in DMEM was heated to 50° C. Immediately prior to overlay, 4% FBS was added to the overlay media, 500 μl of overlay was added to each well, and plates were cultured for 4 days at 37° C., 5% CO₂. At 4 dpi, wells were fixed by adding 1 ml of 10% formaldehyde and incubating at 37° C. overnight. After fixation, media was removed, and monolayers were stained with 1% crystal violet in 20% ethanol for 15 min at room temperature, followed by several rinses with diH₂O and drying before plaques were counted. The plaque reduction neutralization test (PRNT) is considered a “gold standard” in the evaluation of antiviral strategies used against SARS-CoV-2 infections and other viral infections. Using the PLANA-delivered combination of siRNAs targeting coronavirus S-protein and E-protein completely eliminated plaques that were observed in non-treated cell cultures (FIG. 20). In conclusion, we showed the efficiency of an siRNA-based anti-viral approach in vitro in eliminating the damage caused in MRC-5 lung fibroblast cells by hCoV-229E-infection. Administration of siRNA using PLANA nanoparticles by inhalation is a viable approach to the treatment of coronavirus infection, including SARS-CoV-1 (SARS) and SARS-CoV-2 (COVID-19), as well as other respiratory virus infections.

It should be apparent to those skilled in this line of research that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Also, as used in the description herein, the meaning of “in” includes “in” and “on” unless the context clearly dictates otherwise. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety. However, where a definition or use of a term in an incorporated reference is inconsistent or contrary to the definition of that term provided herein, the definition of that term provided herein applies and the definition of that term in the reference does not apply.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A nucleic acid delivery system comprising multi-component peptide lipid-associated nucleic acids (PLANA).

2. The composition of claim 1 when the peptide in PLANA comprises a plurality of positively charged amino acids and a hydrophobic portion, wherein positively charged amino acids and the hydrophobic regions are conjugated to one another.

3. The composition of claim 2 wherein the positively charged amino acids comprise one or more arginine residues.

4. The composition of claim 3, comprising 5 to 6 arginine residues.

5. The composition of claim 2, wherein the hydrophobic portion comprises hydrophobic amino acids.

6. The composition of claim 5, wherein the hydrophobic amino acids constitute a chain attached to the ε-amino group of a lysine residue.

7. The composition of claim 5, wherein the hydrophobic amino acids comprise one or more tryptophan residues.

8. The composition of claim 6, comprising 5 to 7 tryptophan residues.

9. The composition of claim 2, wherein the hydrophobic portion comprises a fatty acyl conjugate.

10. The composition of claim 9, wherein the fatty acyl conjugate is conjugated with the ε-amino group of a lysine residue.

11. The composition of claim 9, wherein the fatty acyl conjugate comprises a C18 chain.

12. The composition of claim 1, wherein the peptide in PLANA is a linear peptide.

13. The composition of claim 1, wherein the peptide in PLANA is a cyclic peptide.

14. The composition of claim 1, wherein the peptide in PLANA is a cyclic/linear hybrid peptide comprising a cyclic positively charged portion having and a linear hydrophobic portion.

15. The composition of claim 1, wherein the lipids comprise cholesterol, phosphatidylcholine, and 1,2-dioleoyl-sn-glycero-3-phosphoethanolamine (DOPE).

16. The composition of claim 15, further comprising 1,2-dioleoyl-3-trimethylammonium-propane (DOTAP).

17. The composition of claim 1, wherein the PLANA is in the form of nanoparticles.

18. A method of transferring a nucleic acid into a living cell, comprising contacting the composition of claim 1 with the cell.

19. A method of making peptide lipid-associated nucleic acids (PLANA) comprising:
a. providing an aqueous solution of nucleic acid,
b. providing an organic solution of lipids,
c. adding a peptide comprising a positively charged portion and a hydrophobic portion to the aqueous solution, and
d. mixing the aqueous and organic solutions in a ratio of 3:1 v/v, to form a PLANA formulation.

20. The method of claim 19, further comprising passing the PLANA formulation through a filter multiple times to form PLANA nanoparticles.


SEQUENCE LISTING (4 sequences)

SEQ ID NO: 1 - RNA, 27 nt, Artificial Sequence
  Forward siRNA targeting the SARS-CoV-2 spike protein
guuaaauuuggcaguguauguuuuucg

SEQ ID NO: 2 - RNA, 26 nt, Artificial Sequence
  Reverse siRNA targeting the SARS-CoV-2 virus spike protein
gaaaaacauacacugccaaauuuaac

SEQ ID NO: 3 - RNA, 27 nt, Artificial Sequence
  Forward siRNA targeting the SARS-CoV-2 virus envelope protein
guuaaauuuggcaguguauguuuuucg

SEQ ID NO: 4 - RNA, 27 nt, Artificial Sequence
  Reverse siRNA targeting the SARS-CoV-2 virus envelope protein
cgaaaaacauacacugccaaauuuaac